(19) United States
(12) Patent Application Publication
(10) Pub. No.: US 2009/0017452 A1
(43) Pub. Date: Jan. 15, 2009

(54) METHODS AND COMPOSITIONS RELATING TO THE PHARMACOGENETICS OF DIFFERENT GENE VARIANTS

(75) Inventors: Mark J. Ratain, Chicago, IL (US); Federico Innoceti, Chicago, IL (US); Deanna L. Kroetz, Burlingame, CA (US); Samir Undevia, Naperville, IL (US); Tan D. Nguyen, San Francisco, CA (US); Wanqing Liu, Chicago, IL (US)

Correspondence Address: FULBRIGHT & JAWORSKI L.L.P., 600 CONGRESS AVE., SUITE 2400, AUSTIN, TX, 78701, US

(73) Assignee: UNIVERSITY OF CHICAGO, Chicago, IL (US)

(21) Appl. No.: 10/591,484

(22) PCT Filed: Mar. 7, 2005
(86) PCT No.: PCT/US2005/007410

§ 371 Date: Oct. 2, 2008

Related U.S. Application Data
(60) Provisional application No. 60/550,268, filed on Mar. 5, 2004.

(51) Int. Cl. C12Q 1/68

(52) U.S. Cl. 435/6

(57) ABSTRACT

The present invention is directed to methods and compositions for determining the presence or absence of polymorphisms within an ABCC2, UGT1A1, and/or SLCO1B1 gene and correlating these polymorphisms with activity levels of their gene products and making evaluations regarding the effect on their substrates, particularly those substrates that are drugs. In addition, there are methods and compositions of evaluating the risk of an individual for developing toxicity or adverse event(s) to an ABCC2, UGT1A1, and/or SLCO1B1 substrate. In some embodiments, the invention concerns methods and compositions for determining the presence or absence of ABCC2 3972C>T variant and predicting or anticipating the level of activity of ABCC2 and determining dosages of an ABCC2 drug substrate, such as irinotecan, in a patient. Such methods and compositions can be used to evaluate whether irinotecan-based therapy, or therapy involving other ABCC2 substrates, may pose toxicity problems if given to a particular patient or predicting their efficacy. Alterations in suggested therapy may ensue based on genotyping results.

The present application claims priority to U.S. Provisional Patent Application Ser. No. 60/550,268, filed on Mar. 5, 2004, which is hereby incorporated by reference in its entirety. The government may own rights in the present invention pursuant to grant number GM61393 from the National Institutes of Health.

### BACKGROUND OF THE INVENTION

1. Field of the Invention

The present invention relates generally to the fields of molecular genetics, pharmacogenetics, and cancer therapy. In particular, the present invention is directed to methods and compositions for detecting polymorphisms and correlating the presence or absence of certain polymorphisms with toxic effects of chemotherapies. More specifically, the present invention is directed to methods and compositions for determining the presence or absence of polymorphisms within an ABCC2 gene, UGT1A1 gene, and/or SLCO1B1 gene, and correlating these polymorphisms with toxic effects of ABCC2 or UGT1A1 substrates, as well as evaluating the risk of an individual for developing toxicity to an ABCC2 or UGT1A1 substrate. In some embodiments, the invention concerns methods and compositions for predicting or anticipating the level of toxicity caused by an ABCC2 or UGT1A1 substrate, such as irinotecan, in a patient. Such methods and compositions can be used to evaluate whether irinotecan-based therapy, or therapy involving other ABCC2 substrates, may pose toxicity problems if given to a particular patient. Alterations in suggested therapy may ensue if a toxicity risk is assessed.

2. Description of Related Art

ATP-binding cassette (ABC) genes represent the largest family of transmembrane proteins that bind ATP and use the energy to drive the transport of various molecules across cell membranes. The products of the ABC genes are known to influence oral absorption and disposition of a wide variety of drugs and play a role in the resistance of malignant cells to anticancer agents (Sparreboom et al., 2000).

ABCC2, a member of the ABC gene family, functions as the major exporter of organic anions from the liver into the bile. In addition, ABCC2 is expressed on the apical membrane of epithelial cells such as enterocytes, renal proximal tubule epithelia, and gall bladder epithelia. ABCC2 is also expressed in some tumor tissues such as ovarian carcinoma, colorectal carcinoma, leukemia, mesothelioma, and hepatocarcinoma; and it has been suggested that tumor cells overexpressing ABCC2 acquire multidrug resistance (MDR) (Borst et al. (1999); Borst et al. (2000)).

ABCC2 substrates include intracellularly formed glucuronide and reduced glutathione (GSH)—conjugates of clinically important drugs (Suzuki et al., 1998). In addition, ABCC2 is also involved in the biliary excretion of non-conjugated anionic drugs such as irinotecan (CPT-11).

Irinotecan is an antineoplastic drug used in the treatment of colon cancer. Irinotecan hydrolysis by carboxylesterase-2 (CES-2) is responsible for its activation to SN-38 (7-ethyl-10-hydroxycamptothecin), a topoisomerase I inhibitor of much higher potency than irinotecan. The main inactivating pathway of irinotecan is the biotransformation of active SN-38 into inactive SN-38 glucuronide (SN-38G) by UDP-glucuronosyltransferase 1A1 (UGT1A1) (Iyer et al., 1998).

Hepatic glucuronidation results from the activities of a multigene family of UGT enzymes, the members of which exhibit specificity for a variety of endogenous substrates and xenobiotics. The UGT enzymes are broadly classified into two distinct gene families. The UGT1 locus codes for multiple isoforms of UGT, all of which share a C-terminus encoded by a unique set of exons 2-5, but which have a variable N-terminus encoded by different first exons, each with its own independent promoter (Bosma et al., 1992; Ritter et al., 1992). The variable first exons confer the substrate specificity on the enzyme. Isoforms of the UGT2 family are unique gene products of which at least eight isozymes have been identified. (Clarke et al., 1994). The UGT1A1 isoform is the major bilirubin glucuronidation enzyme. Genetic defects in the UGT1A1 gene can result in decreased glucuronidation activity which leads to abnormally high levels of unconjugated serum bilirubin that may enter the brain and cause encephalopathy and kernicterus; Owens & Ritter, (1995). As described above, this condition is commonly known as Gilbert's syndrome (which is frequently diagnosed based on elevated total bilirubin levels—a biochemical diagnosis). The molecular defect in Gilbert's Syndrome is a change in the TATA box within the UGT1A1 promoter (Bosma et al. 1995; Monaghan et al, 1996). This promoter usually contains a (TA)₆ TAA element, but another allele, termed UGT1A1*28 or allele 7, is also present in human populations at high frequencies, and contains the sequence (TA)₇ TAA. This polymorphism in the promoter of the UGT1A1 gene results in reduced expression of the gene and accounts for most cases of Gilbert's Syndrome (Bosma et al., 1995). As discussed below, overall, gene expression levels for the UGT1A1 promoter alleles are inversely related to the length of the TA repeat in the TATA box.

The variation observed in this promoter may also account for the inter-individual and inter-ethnic variations in drug metabolism and response to xenobiotic exposure. UGTs have been shown to contribute to the detoxification and elimination of both exogenous and endogenous compounds, including irinotecan. Examples of how UGT polymorphisms affect irinotecan can be found in U.S. Pat. Nos. 6,472,157 and 6,395,481, which are both incorporated by reference with respect to their teaching about UGT1A1 sequences and TA repeats.

Despite its efficacy in treating metastatic colon cancer and its broad spectrum of activity in other tumor types, irinotecan treatment is associated with significant toxicity. The main severe toxicities of irinotecan are delayed diarrhea and myelosuppression. Moreover, a number of patients develop neutropenia, a blood disorder, as a result of treatment. In the early single agent trials, grade 3-4 diarrhea occurred in about one third of patients and was dose limiting (Negoro et al., 1991; Rothenberg et al., 1993). Its frequency varies from study to study and is also schedule dependent. The frequency of grade 3-4 diarrhea in the three-weekly regimen (19%) is significantly lower compared to the weekly schedule (36%, Fuchs et al., 2003). In addition to diarrhea, grade 3-4 neutropenia is also a common adverse event, with about 30-40% of the patients experiencing it in both weekly and three-weekly regimens (Fuchs et al., 2003; Vanhoefer et al., 2001). Fatal events during irinotecan treatment have been reported. A high mortality rate of 5.3% and 1.6% was reported in the weekly and three-weekly single agent irinotecan regimens, respectively (Fuchs et al., 2003).

Interpatient differences in systemic formation of SN-38G have been shown to have clear clinical consequences in patients treated with irinotecan. Patients with higher glucuronidation of SN-38 are more likely to be protected from the dose limiting toxicity of diarrhea in the weekly schedule (Gupta et al., 1994).

Improved methods and compositions for the evaluation of risk for irinotecan toxicity in an individual are still needed. Clearance of irinotecan and its metabolites by ABCC2 represents a mechanism to protect patients from the toxic effects. However, the problem of identifying the effects of various polymorphisms on drug clearance by ABCC2 remains. Resolving these problems would provide novel methods and compositions for the evaluation of risk for toxicity to irinotecan as well as for numerous other drugs that are substrates for ABCC2.

### SUMMARY OF THE INVENTION

The present invention is based on identification and characterization of correlations between genotype of the ABCC2 gene and phenotype relating to the activity of ABCC2. Thus, the present invention provides methods and compositions that exploit correlations between genotype and phenotype concerning ABCC2. The present invention also concerns a correlation between the genotype and phenotype of other genes whose gene products affect substrates of ABCC2. These other genes include the UGT1A1 gene and the SLCO1B1 gene. Therefore, the present invention also relates to methods and compositions involving polymorphisms in these genes as well and the ramifications of those polymorphisms on the effects of particular drugs in certain patients.

It is contemplated that such methods and compositions have diagnostic, prognostic, and therapeutic applications.

The present invention involves methods for determining the level of ABCC2 activity in a patient. This method can be used to predict what the level of ABCC2 activity is in a patient based on genotypic analysis.

In some embodiments, the method involves a) determining the sequence at position 3972 in one or both alleles of the ABCC2 gene of the patient, wherein a C at position 3972 on one or both alleles is indicative of a normal level of ABCC2 activity.

Additional methods of the invention include a method for predicting tumor response to an anticancer agent that is an ABCC2 substrate in a cancer patient comprising a) determining the sequence at position 3972 in one or both alleles of the ABCC2 gene of the patient, wherein a C at position 3972 on one or both alleles is indicative of a greater chance of a reduced antitumor response to the anticancer agent. The probability of a reduced antitumor response is increased with respect to persons who do not have a C at position 3972. The determination of a T on both alleles at position 3972 in the ABCC2 gene is indicative of a greater chance of an antitumor response or of a better antitumor response than would be expected as compared to a person with a C at position 3972.

The term “antitumor response” means a response that results in a favorable therapeutic outcome with respect to a tumor. Examples of such an outcome include, but are not limited to, reduction in tumor size, retardation of tumor growth or proliferation, inhibition of metastasis, reduction in number of metastasis, inhibition of tumor vasculature, inhibition of tumor growth rate, promotion of apoptosis of tumor cells, induction of tumor cell death or killing, promotion of remission of cancer growth, and extended survival. Thus, a reduced antitumor response means the patient may exhibit no response to the drug or that the response is less favorable than would be expected for someone with a TT genotype at position 3972. It will understood that the prediction of a reduced antitumor response may lead to an increased dosage (increased concentration, increased administration frequency and/or both) and/or more aggressive treatment regimen than would have been the case for someone with the TT genotype. This altered treatment may overcome the predicted reduced antitumor response. Thus, embodiments of the invention further include adjusting dosage (concentration and/or administration (timing and/or frequency)) or route of administration of the anticancer agent or altering the treatment regimen overall. In some cases, the time between treatment regimens may be altered. In specific embodiments, the anticancer agent is irinotecan.

Other methods of the invention concern a method for determining dosage of an ABCC2 substrate for a patient comprising: a) determining the sequence at position 3972 in one or both alleles of the ABCC2 gene of the patient, wherein a C at position 3972 on one or both alleles indicates a higher dosage of the substrate than is indicated for a patient with a T at position 3972 in both alleles of the ABCC2 gene.

The present invention also concerns a method for predicting a clearance rate for irinotecan in a patient. The method involves a) determining the sequence at position 3972 in one or both alleles of the ABCC2 gene of the patient, wherein a C at position 3972 in one or both alleles is indicative of a normal clearance rate for irinotecan. Again, “normal” is with respect to the level of clearance that is expected for persons with the TT haplotype at position 3972. In additional embodiments, the clearance rate is determined empirically in that patient based on techniques that are well known to those of skill in the art. Identification of a T at position 3972 on both alleles of the ABCC2 gene is indicative of a lower than normal clearance rate for irinotecan. In specific embodiments, it is contemplated that a method for predicting a clearance rate for irinotecan in a patient comprises: a) determining the sequence of the patient at either i) position 3972 in one or both alleles of the ABCC2 gene, wherein a C at position 3972 in one or both alleles is indicative of a normal clearance rate for irinotecan; ii) position 521 in one or both alleles of the SLCO1B1 gene, wherein a C at position 521 in one or both alleles is indicative of a lower clearance rate than a T in both alleles; or iii) both positions i) and ii). The presence of a T in both alleles at position 521 in the SLCO1B1 gene is indicative of a higher clearance rate than a C at that position in one or both alleles. It is also contemplated that clearance rate may be assessed after a patient has taken the drug, and further refinements in the regimen of the drug are made with respect to the patient's intake.

Methods of the present invention can also be employed to predict the risk of irinotecan toxicity in a patient comprising: a) determining the sequence at position 3972 in one or both alleles of the ABCC2 gene of the patient, wherein a C at position 3972 indicates a lower risk of toxicity than a T at position 3972 in both alleles of the ABCC2 gene. Toxicity is evidenced in patients by a number of ailments, including diarrhea and neutropenia.

In some embodiments, any of the methods of the invention discussed herein includes, either in addition to or instead of step a) one or more of the following steps: b) determining the number, if any, of haplotype 4 in the ABCC2 gene (−1549 A, −1019 G, −24 C, 1249 G, 34 T in intron 27, and 3972 T) of the patient, wherein one allele of haplotype 4 is indicative of a greater risk of toxicity than for a patient having two alleles with haplotype 4 but a lesser risk of toxicity than for a patient having no alleles with haplotype 4; and/or c) determining the sequence in one or both alleles of the SLC01B1 gene at position 388, wherein i) a G in one allele is indicative of a similar or lower risk than an A in one allele, or ii) a G in both alleles is indicative of a lower risk than a G in one allele and an A in the other allele, which is indicative of a lower risk than an A in both alleles. In other embodiments, it is contemplated that methods may be implemented with a), b), and/or c), and that any methods may further comprise: d) determining the sequence in one or both alleles of the UGT1A1 gene at position −3156, wherein i) a G in one allele is indicative of a similar or lower risk than an A in one allele, or ii) a G in both alleles is indicative of a lower risk than a G in one allele and an A in the other allele, which is indicative of a lower risk than an A in both alleles; and/or, e) determining the number of TA repeats in the promoter of the UGT1A1 gene, wherein i) six TA repeats in one allele is indicative of a similar or lower risk than seven TA repeats in one allele, or ii) six TA repeats in both alleles is indicative of a lower risk than six TA repeats in one allele and seven TA repeats in the other allele, which is indicative of a lower risk than seven TA repeats in both alleles.

Whether a patient has haplotype 4 and the number that a patient has in his/her ABCC2 gene are correlated with toxicity of ABCC2 drug substrates. Haplotype 4 means having the following genotype with respect to the ABCC2 gene: −1549 A, −1019 G, −24 C, 1249 G, 34 T in intron 27, and 3972 T, meaning the patient has the specified sequence at the specified position. A patient having two alleles with haplotype 4 has a lower risk of toxicity than a patient with one haplotype 4 allele. A patient with one haplotype 4 allele is predicted to have a lower risk than a patient who does not have haplotype 4. In other words, having one allele of haplotype 4 is indicative of a greater risk of toxicity than for a patient having two alleles with haplotype 4 but a lesser risk of toxicity than for a patient having no alleles with haplotype 4. The correlation of risk with number of haplotype 4, from lowest to highest, is: 2, 1, 0.

Identifying the sequence at position 388 of the SLC01B1 gene provides information regarding toxicity issues. Having a G in one allele is indicative of a similar or lower risk than having an A in one allele. Having a G in both alleles is indicative of a lower risk than having a G in one allele and an A in the other allele, which is indicative of a lower risk than an A in both alleles. In other words, the correlation of risk at position 388 of the SLC01B1 gene, from lowest to highest is: G/G, A/G, A/A.

The sequence of the UGT1A1 gene at position −3156 is relevant because i) a G in one allele is indicative of a similar or lower risk than an A in one allele, and ii) a G in both alleles is indicative of a lower risk than a G in one allele and an A in the other allele, which is indicative of a lower risk than an A in both alleles. The correlation of risk at position −3156 of the UGT1A1 gene, from lowest to highest is: G/G, A/G, A/A.

The number of TA repeats (also referred to as (TA)ₙ) in the promoter of the UGT1A1 gene has been correlated with drug toxicity previously. Six TA repeats in one allele is indicative of a similar or lower risk than seven TA repeats in one allele. Six TA repeats in both alleles is indicative of a lower risk than six TA repeats in one allele and seven TA repeats in the other allele, which is indicative of a lower risk than seven TA repeats in both alleles. The correlation of risk with the number of TA repeats in the promoter of the UGT1A1 gene, from lowest to highest is: 6/6, 6/7, 7/7. Relatively few patients have an allele in which the number of TA repeats is 5 or 8.

In some embodiments, the ABCC2 substrate is selected from the group of substrates consisting of cysteinyl leukotrienes, glutathione and glutathione conjugates, glucuronide conjugates, sulfated conjugates, bile salt conjugates, bromosulfophthalein, and dibromosulfophthalein (see Table 1). Identified in Table 1 are substrates that are administered as drugs to patients. Determining the dosage of any of these drugs is specifically contemplated as part of the invention. In some cases, the dosage that would be given to a patient is modified based on the genotyping results based on methods of the invention. In certain embodiments, the substrate is irinotecan, SN-38, APC, and/or SN-38G. Methods of the invention also include prescribing a dosage of the anticancer agent, such as irinotecan, based on the determination of the sequence at position 3972 in one or both alleles of the ABCC2 gene. It is contemplated that a patient is given a different dosage than he or she would have otherwise received had the genotyping not been performed. Thus, in some embodiments of the invention, a typical dosage is adjusted for a particular person (individualized therapy).

It is contemplated that the invention is not limited to ABCC2 substrates and can include UGT1A1 substrates. Embodiments involving ABCC2 substrates may be applied with respect to a UGT1A1 substrate.

In certain embodiments, assessments will involve also considering other factors such as total bilirubin amounts in the patient and gender. Evidence indicates that drug toxicity, such as from irinotecan, is more prevalent among females than males. Thus, in some embodiments of the invention, the methods also include assaying total bilirubin amounts in the patient.

It will be of course understood that the assessments or predictions of activity and response are relative with respect to patients having a different genotype at the relevant position(s). Moreover, when multiple polymorphisms or factors are considered the effect will be considered additive with respect to those indicators that identify a greater or higher risk of toxicity. A person of ordinary skill in the art will use these different indicators in considering adjustments in dosage that might reduce the risk of toxicity in the patient.

Methods of the invention also include monitoring for toxicity or adverse events once the ABBC2 substrate is administered, and possibly, adjusting or modifying dosage based on those results. Toxicity indicators or indicators of adverse events include diarrhea, neutropenic fever, other hematologic toxicities, as well as known non-hematologic toxicities.

Reference to nucleotides (or residues) may be according to their well known abbreviations. A “C” refers to a cytosine; “T” refers to “thymine”; “A” refers to adenine; and, “G” refers to guanine. If mRNA is used to determine a nucleotide sequence, “U” refers to uracil. In one study, the allele frequency for the variant allele (T) at position 3972 was 38.3% in Caucasians (n=100) and 27.3% African Americans (n=100). It is understood that a C is the most common nucleotide at position 3972. Because of that and the observations discussed herein, the activity of ABCC2 will be characterized relative to the activity of ABCC2 in persons with a C at 3972. Consequently, a normalized level of activity of ABCC2 in persons with a C at 3972 will be understood as a “normal level of ABCC2 activity.” Moreover, in some embodiments of the invention, identification of a T at position 3972 on both alleles of the ABCC2 gene is indicative of a lower than normal level of ABCC2 activity.

It will be understood that the term “determine” is used according to its ordinary and plain meaning to indicate “to ascertain definitely by observation, examination, calculation, etc.,” according to the Oxford English Dictionary (2^{nd} ed.). It will also be understood that the phrase “determining the sequence at position X” means that the nucleotide at that position is directly or indirectly identified. In some embodiments, the sequence at a particular position is determined, while in other embodiments, what is determined at a particular position is that a particular nucleotide is not at that position.

Positions are indicated by conventional numbering where a negative sign (−) refers to nucleotides upstream (5′) from the transcriptional start site (+1) (these sequences are in the promoter), unless otherwise designated. A sequence in the 5′ untranslated region (5′ UTR) may also be referred to by a negative sign, and in these cases, the positioning is with respect to the translated portion, where the first nucleotide of a codon is understood as +1. Positions downstream of the translational start site may or may not have a plus sign (+). Furthermore, unless otherwise indicated or understood, identification of a position downstream of the transcriptional start site refers to a position with respect to only the coding region of the gene, that is, its exons and not the introns. In some instances, positions within introns are referred to and the numbering for these positions is typically with respect to that intron alone, and not the gene as a whole.

It is contemplated that in methods of the invention, one or more sequences in one or both alleles of the ABCC2 gene is determined. This is also the case with respect to other polymorphisms in other genes, such as the UGT1A1 gene and the SLCO1B1 gene. In some embodiments, both alleles of the patient are evaluated, while in others, only one allele is evaluated.

In further embodiments of the invention, methods also include obtaining a sample from a patient and using the sample to determine one or more sequences or to evaluate haploytpe or number of TA repeats. The sample may contain blood, serum, or a tissue biopsy, as well as buccal cells, mononuclear cells, or cancer cells.

Sequences may be determined by performing or conducting a hybridization assay, an amplification assay, particularly one that is allele-specific, a sequencing or microsequencing assay.

Determining sequence, whether a patient has a particular haplotype and how many, and the number of TA repeats in the UGT1A1 promoter, may be determined directly or indirectly. A direct determination involves performing an assay with respect to that position(s). An indirect determination means that a determination is based on data regarding a different position, particularly by evaluating the sequence of a position in linkage disequilibrium (LD) with the sequence, haplotype or number of TA repeats. For example, an indirect determination of the sequence at position 3972 of the ABCC2 gene can involve identifying the sequence of a position in LD with position 3972. In some embodiments, the sequence in LD with a sequence at position 3972 is in complete linkage disequilibrium with a sequence at 3972. In additional embodiments, the position in linkage disequilibrium with the sequence at position 3972 of the ABCC2 gene is selected from the group consisting of positions −1549 (promoter), −1019 (promoter), −24 (5′ UTR), and +27 (intron 13) in the ABCC2 gene. In some cases, more than one position in linkage disequilibrium with the sequence, haplotype, or number of TA repeats is evaluated. Therefore, in some embodiments of the invention, a haplotype that includes position 3972 is evaluated. In these embodiments, a determination of one or more sequences in one or both alleles of a gene in the haploytpe is included in methods of the invention.

In methods of the invention, in some embodiments, an additional step of administering an ABCC2 substrate to the patient is included. Likewise, in some embodiments, the step of administering an anticancer agent to the patient is included in methods of the invention. In some cases, the amount, formulation, or timing of the administration is based on the genotypic analysis discussed above. In some embodiments of the invention, a patient is also provided additional anticancer therapy, such as the administration of a second anticancer agent or the performance of surgery on the patient. The second anticancer agent may be chemotherapy, particularly one that is not an ABCC2 substrate or not the same ABCC2 substrate that was already given to the patient, radiation therapy, immunotherapy, or gene therapy. In specific embodiments, the ABCC2 substrate is irinotecan.

The present invention further concerns compositions that can be used to determine the sequence at position 3972 or any other sequence in LD with it. Furthermore, it concerns compositions that can be used to identify any sequence discussed herein or determine the number of either TA repeats or haplotypes. Accordingly, the present invention concerns kits for achieving methods of the invention. It is contemplated that kits can include particular components in suitable containers for uses consistent with the invention.

In some embodiments, the kits include one or more nucleic acids for determining the sequence at position 3972 in at one or both alleles of the ABCC2 gene. In certain embodiments, the present invention concerns a kit comprising at least one nucleic acid for determining the sequence at a) position 3972, 1549, −1019, −24, 1249, 34 in intron 27, and/or 3972 in an ABCC2 gene; and/or b) position 388 in a SLC01B1 gene. In additional embodiments, the kit may also include at least one nucleic acid for determining: c) the sequence at position −3156 in a UGT1A1 gene; and/or d) the number of TA repeats in the UGT1A1 gene promoter. Thus, it is contemplated that kits of the invention can include one or more nucleic acids for determining the sequence at any of the 10 polymorphisms discussed above. In certain embodiments, nucleic acids for determining the sequence of 1, 2, 3, 4, 5, 6, 7, 8, 9, or 10 polymorphisms, or any range derivable therein, can be included. In certain embodiments, the kit comprises nucleic acids for detecting for the presence of haplotype 4. Moreover, ckit components can include nucleic acids derived from SEQ ID NO: 1 and/or SEQ ID NOs:3-11.

In some embodiments, the nucleic acid is a primer for amplifying the sequence. In others, the nucleic acid is a specific hybridization probe for detecting the sequence. A probe can also be adjacent to the specific hybridization probe for a sequence. Additionally, it is contemplated that the specific hybridization probe can be comprised in an oligonucleotide array or microarray.

It is contemplated that any method or composition described herein can be implemented with respect to any other method or composition described herein. Similarly, any embodiment discussed with respect to one aspect of the invention may be used in the context of any other aspect of the invention.

Throughout this application, the term “about” is used to indicate that a value includes the standard deviation of error for the device or method being employed to determine the value.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternative are mutually exclusive, although the disclosure supports a definition that refers to only alternatives and “and/or.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: ABCC2 3972C>T variant and AUC values of irinotecan and APC.

FIG. 2: ABCC2 3972C>T variant and AUC values of SN-38 and SN-38G.′

FIG. 3. Haplotype structure of ABCC2 gene.

FIG. 4. SN38 AUC Box Plot against occurrence of Haplotype 4.

FIG. 5. Log(ANC) mapping of patients showing those with 0, 1, or 2 (TA)₆ and number of ABCC2 haplotype 4 (0, 1, or 2).

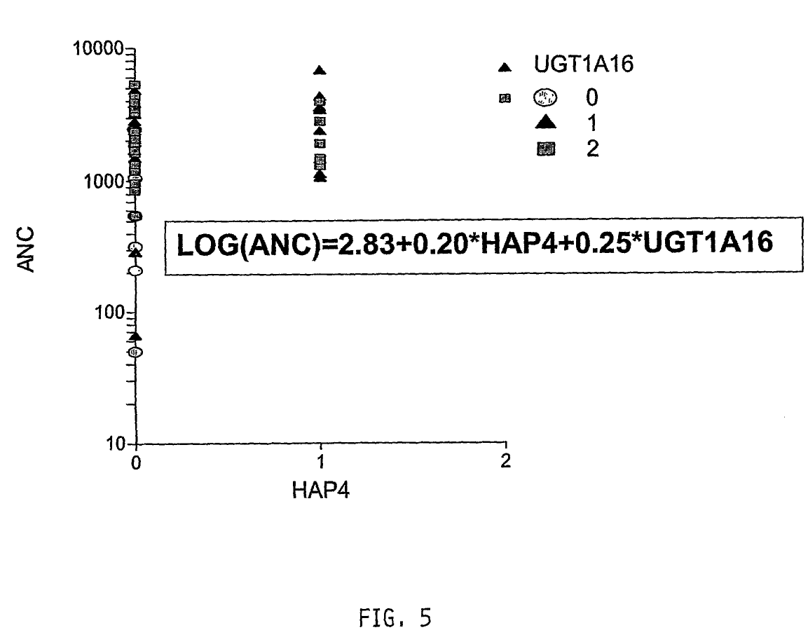

### DESCRIPTION OF ILLUSTRATIVE EMBODIMENTS

The present invention provides improved methods and compositions for identifying the effects of polymorphisms in ABCC2 on the disposition of drugs and drug metabolites for the evaluation of the potential risk for drug toxicity or adverse events in an individual or patient. The development of these improved methods and compositions allows for the use of such an evaluation to optimize treatment of a patient and to lower the risk of toxicity or adverse events.

One particular ABCC2 drug substrate is irinotecan, a chemotherapeutic used in the treatment of cancer. Irinotecan is also inactivated to oxidated metabolites (including APC) by CYP3A enzymes, and is activated to SN-38, which has a 100-1,000-fold higher antitumor activity than irinotecan, by carboxylesterase-2 (CES-2). SN-38 is glucuronidated by hepatic uridine diphosphate glucuronosyltransferases (UGTs) to form SN-38 glucuronide (10O-glucuronyl-SN-38, SN-38G), which is inactive and excreted into the bile and urine although, SN-38G might be deconjugated to form SN-38 by intestinal β-glucuronidase enzyme (Kaneda et al., 1990). Irinotecan, SN-38, and SN-38G are known substrates for ABCC2. (Suzuki et al. (1999); Suzuki et al. (1998)).

The major dose-limiting toxicities of irinotecan include diarrhea and, to a lesser extent, myelosuppression. Irinotecan-induced diarrhea can be serious and often does not respond adequately to conventional antidiarrheal agents (Takasuna et al., 1995). This diarrhea may be due to direct enteric injury caused by the active metabolite, SN-38, which has been shown to accumulate in the intestine after intra peritoneal administration of irinotecan in athymic mice (Araki et al., 1993). In addition to diarrhea, grade 3-4 neutropenia is also a common adverse event, with about 30-40% of the patients experiencing it in both weekly and three-weekly regimens (Fuchs et al. 2003; Vanhoefer et al., 2001). Fatal events during irinotecan treatment have been reported. A high mortality rate of 5.3% and 1.6% was reported in the weekly and three-weekly single agent irinotecan regimens, respectively (Fuchs et al., 2003).

It has been shown that there is an inverse relationship between SN-38 glucuronidation rates and severity of diarrheal incidences in patients treated with increasing doses of Irinotecan (Gupta et al., 1994). These findings indicate that glucuronidation of SN-38 protects against Irinotecan-induced gastrointestinal toxicity. Therefore, differential rates of SN-38 glucuronidation among subjects may explain the considerable inter-individual variation in the pharmacokinetic parameter estimates and toxicities observed after treatment with anti-cancer drugs or exposure to xenobiotics (Gupta et al., 1994; Gupta et al, 1997).

In addition to the genes discussed below, other factors that appear to play a role in irinotecan toxicity issues are total amounts of bilirubin in the plasma and gender. Methods of assessing total amounts of bilirubin can be found in U.S. Pat. No. 5,786,344, which is herein incorporated by reference. The amount of total bilirubin correlates with a risk for toxicity such that a higher amount correlates with a higher risk. An amount of bilirubin in plasma that is greater than 1.0-1.2 mg/dl is indicative for a risk of toxicity. An amount of bilirubin in plasma that is greater than 3 mg/dl (about 50 μM) is indicative of a significant risk of toxicity. Also, females are at greater risk of experiencing irinotecan toxicity than males.

### I. ABCC2

ABCC2, also referred to as MRP2 and cMOAT, functions as the major exporter of organic anions from the liver into the bile (SEQ ID NO:2 is protein sequence). In addition, ABCC2 is expressed on the apical membrane of epithelial cells such as enterocytes, renal proximal tubule epithelia, and gall bladder epithelia. ABCC2 is also expressed in some tumor tissues such as ovarian carcinoma, colorectal carcinoma, leukemia, mesothelioia, and hepatocarcinoma; and it has been suggested that tumor cells overexpressing ABCC2 acquire multidrug resistance (MDR) (Borst et al. (1999); Borst et al. (2000)).

ABCC2 is important from a pharmacological point of view because it is involved in the clearance of several clinically important drugs. One such drug is the anticancer drug irinotecan (CPT-11).

The present invention demonstrates that the synonymous 3972C>T (exon 28) in ABCC2 is correlated with AUC (area under the curve) for irinotecan (p=0.02), APC (p=<0.0001), APC/irinotecan ratio (p=<0.0001), SN-38G (p≦0.001), and SN-38G/SN-38 (p≦0.001). Furthermore, the TT 3972 genotype was associated with higher AUC of irinotecan (p=0.02), APC (p<0.0001), and SN-38G (p<0.0001) compared to CT and CC patients. The phenotypic effect of 3972C>T was previously unknown, and identifies 3972C>T as a variant potentially affecting ABCC2 activity and suggests its biological function and clinical relevance for ABCC2 substrates.

Other data also reveal that a particular haplotype for ABCC2 is relevant to drug toxicity. Haplotype 4, which is defined as −1549A, −1019G, −24C, 1249G (Exon 10), Intron 27 34T, and 3972T (Exon 28). Note that numbering for introns is with respect to that particular intron. The 5′ noncoding sequence of the ABCC2 gene can be found at GenBank Accession No. AF144630 (SEQ ID NO:10), which is hereby incorporated by reference. A 3′ portion of the noncoding sequence of the ABCC2 gene discussed above can be found at GenBank Accession No. AL392107, which is hereby incorporated by reference. The exons for ABCC2 have been mapped. For example, exon 27 is found at AJ132309 and exon 28 is found at AJ132310. The sequence for intron 27 can be found in SEQ ID NO:11, which shows nucleic acid residues 33456 to 35264 of AL392107. The beginning of intron 27 is from 33456 and the end of the intron is from 35164. Position 34 of intron 27 is at 35131 and is shown in the corresponding position in SEQ ID NO:11.

Thus, the present invention provides improved methods and compositions for evaluating the disposition of drugs and drug metabolites, and for evaluating the potential risk for drug toxicity in an individual or patient. The development of these improved methods and compositions allows for the use of such an evaluation to optimize treatment of a patient and to lower the risk of toxicity.

AUC is a measure of how much drug reaches the bloodstream in a set period of time. AUC is calculated by plotting drug blood concentration at various times over a specified period of time, usually 24 hours, and then measuring the area under the curve. AUC has an number of important uses in toxicology, biopharmaceutics, and pharmacokinetics. It is understood to be the time course or exposure of the patient to the drug.

The metabolism of irinotecan is merely illustrative of the present invention; the metabolism of other ABCC2 substrates is also contemplated. A summary of ABCC2 substrates is provided in Table 1 below. The table includes ABCC2 drug substrates.

TABLE 1
ABCC2 Substrates

Cysteinyl Leukotrienes
LTC₄
LTD₄
LTE₄
N-acetylated LTE₄
GSH and GSH-Conjugates of Organic Compounds
Reduced glutathione (GSH)
Oxidized glutathione (GSSG)
2,4-dinitrophenol-S-glutathione
Glutathione-bimane
GSH Conjugate of bromosulfophthalein
GSH Conjugate of bromoisovalerylurea
GSH Conjugate of N-ethylmaleimide
GSH Conjugate of ethacrynic acid
GSH Conjugate of α-naphthylisothiocyanate
GSH Conjugate of methylfluoroscein
GSH Conjugate of prostaglandin A1
GSH Conjugate of (+)-anti-benzo[a]pyrene-7,8-diol-9,10-epoxide
GSH Conjugate of 4-hydroxynonenal
GSH Conjugates of Metals
Antimony
Arsenic
Bismuth
Cadmium
Copper
Silver
Zinc
Glucuronide Conjugates
Bilirubin monoglucuronide
Bilirubin diglucuronide
17β estradiol 17β-D-glucuronide
Triiodothyronine-glucuronide
p-nitrophenol-β-D-glucuronide
1-naphytol-β-D-glucoronide
E3040 glucuronide
SN-38 glucuronide (SN-38G)
Grepafloxacin glucuronide
4-(methylnitrosoamino)-1-(3-pyridyl)-1-butanol glucuronide
Telmisaltan glucuronide
Acetaminophen glucuronide
Diclofenac glucuronide
Indomethacin glucuronide
Glucuronide conjugates of 2-amino-1-methyl-6-
phenylimidazo[4,5-b]pyridine
Liquiritigenin glucuronide
Glycyrrhizin
Sulfated Conjugates
Dehydroepiandrosterone sulfate
Bile Salt Conjugates
Cholate-3-O-glucuronide
Lithocholate-3-O-glucuronide
Chenodeoxycholate-3-O-glucuronide
Nordeoxycholate-3-O-glucuronide
Nordeoxycholate-3-sulfate
Lithocholate-3-sulfate
Taurolithocholate-3-sulfate
Glycolithocholate-3-sulfate
Taurochenodeoxycholate-3-sulfate
Non-Conjugated Compounds
Bromosulfophthalein
Dibromosulfophthalein
Carboxyfluorescein
Reduced folates
Methotrexate
CPT-11
SN-38
Ampicillin
Ceftriaxone
Cefodizime
Grepafloxacin
Pravastatin
Temocaprilat
BQ123
p-aminohippuric acid
Fluo-3
Sulfinpyrazone (GSH coupled)
Vinblastine (GSH coupled)
2-amino-1-methyl-6-phenylimidazo[4,5-b]pyridine (GSH coupled)
Etoposide
Vincristine
Doxorubicin
Epirubicin
Cisplatin

### II. UGT1A Enzymes

Glucuronidation plays a major role in the pharmacological activity and clearance of a large variety of compounds (Tukey and Strassburg, 2000). Genetic studies of UDP-glucuronosyltransferases (UGTs) aim to characterize an individual's predisposition to various diseases and increased risk of adverse outcome to drug treatment. The variation in the UDP-glucuronosyltransferase 1A1 (UGT1A1) gene is the most extensively studied. The UGT1A1 gene sequence can be found at GenBank Accession No. AF279093, which is hereby incorporated by reference. UGT1A1 basal expression is affected by the variable number of TA repeats in the TATA box, i.e., (TA)ₙ, see U.S. Pat. No. 6,395,481, which is incorporated herein by reference. A variable number of repeats (5, 6, 7, and 8) have been found in the UGT1A1 TATA box. Gene transcriptional efficiency has been inversely correlated to the number of TA repeats (Beutler et al., 1998). Thus, a larger TA repeat number is associated with reduced transcriptional activity (Beutler et al., 1998) leading to various degrees of impaired glucuronidation of UGT1A1 substrates. The sequence for number of TA repeats is found in SEQ ID NO:5 (five repeats); SEQ ID NO:6 (six repeats); SEQ ID NO:7 (seven repeats); and, SEQ ID NO:8 (eight repeats). Moreover, a polymorphism at −3156 in the UGT1A1 promoter was found in sequence disequilibrium with the number of TA repeats. See U.S. Pat. App. Publication No. 20040203034, which is hereby incorporated by reference for teachings regarding UG1A1 polymorphisms and irinotecan toxicity and methods of evaluating such polymorphisms.

Homozygosity for (TA)₇ allele is associated with Gilbert's syndrome (a familial mild hyperbilirubinemia) (Bosma et al., 1995 and Monaghan et al. 1996) and predisposition to the toxic effects of cancer treatment with irinotecan (Ando et al., 2000 and Iyer et al., 2002). Gilbert's syndrome has also been associated with missense coding variants in the UGT1A1 gene, in particular in Asian populations where these variants are relatively common. Increased risk of breast cancer was reported in African-American women who carried the (TA), and (TA)₈ alleles (Guillemette et al., 2000). In addition to the TATA box, Sugatani et al., (2001) identified a region in the UGT1A1 promoter approximately 3 kb upstream of the TATA box that regulates UGT1A1 inducibility by phenobarbital. It is also hypothesized that this phenobarbital-responsive enhancer module (PBREM) might be modulated by endogenous factors (Sugatani et al. 2002). UGT1A1 activity is probably the result of PBREM-dependent modulation of TATA box-dependent basal expression.

Irinotecan hydrolysis by carboxylesterase-2 is responsible for its activation to SN-38 (7-ethyl-10-hydroxycamptothecin), a topoisomerase I inhibitor of much higher potency than irinotecan. The main inactivating pathway of irinotecan is the biotransformation of active SN-38 into inactive SN-38 glucuronide (SN-38G). Interpatient differences in systemic formation of SN-38G have been shown to have clear clinical consequences in patients treated with irinotecan. Patients with higher glucuronidation of SN-38 are more likely to be protected from the dose limiting toxicity of diarrhea in the weekly schedule (Gupta et al., 1994). SN-38 is glucuronidated by UDP-glucuronosyltransferase 1A1 (UGT1A1) (Iyer et al., 1998).

### III. SLCO1B1

The solute carrier organic anion transporter family member 1B1, SLCO1B1 (also known as organic anion transporting polypeptide-C or OATP-C) has only recently been studied for a correlation between polymorphisms and pharmacokinetics. In a study involving prevastin, a correlation was observed.

As described in the paper of Niemi et al., 2004, which is hereby incorporated by reference, the SLCO1B1 gene was sequenced completely in all subjects. Of the six outliers evaluated, five were heterozygous for the SLCO1B1 521T>C (Val174Ala) SNP (allele frequency 42%) and three were heterozygous for a new SNP in the promoter region of OATP-C (−11187G>A, allele frequency 25%). Among the remaining 35 subjects, two were homozygous and six were heterozygous carriers of the 521T>C SNP (allele frequency 14%, P=0.0384 versus outliers) and three were heterozygous carriers of the −11187G>A SNP (allele frequency 4%, P=0.0380 versus outliers). In subjects with the −11187GA or 521TC genotype, the mean pravastatin AUC0-12 was 98% (P=0.0061) or 106% (P=0.0034) higher, respectively, compared to subjects with the reference genotype. These results were substantiated by haplotype analysis. In heterozygous carriers of *15B (containing the 388A>G and 521T>C variants), the mean pravastatin AUCO-12 was 93% (P=0.024) higher compared to non-carriers and, in heterozygous carriers of *17 (containing the −11187G>A, 388A>G and 521T>C variants), it was 130% (P=0.0053) higher compared to non-carriers.

Others have begun investigating this gene role in irinotecan toxicity (Nozawa et al. 2005, which is hereby incorporated by reference). HEK293 cells stably transfected with SLCO1B1*1a (OATP-C*1a) coding wild-type OATP1B1 were used. The effect of single nucleotide polymorphisms in OATP1B1 was evaluated by measuring uptake activity in Xenopus oocytes expressing OATP1B1*1a and three common variants. In all cases, transport activity for SN-38 was observed, whereas irinotecan and SN-38G were not transported. Moreover, SN-38 exhibited a significant inhibitory effect on SLCO1B1-mediated uptake of [(3)H]estrone-3-sulfate. Among the variants examined, SLCO1B1*15 (N130D and V174A; reported allele frequency 10-15%) exhibited decreased transport activities for SN-38 as well as pravastatin, estrone-3-sulfate, and estradiol-17beta-glucuronide.

The coding sequence for SLCO1B1 is SEQ ID NO:9, which is GenBank Accession No. NM 006446, hereby incorporated by reference.

### IV. Nucleic Acids

Certain embodiments of the present invention concern various nucleic acids, including amplification primers, oligonucleotide probes, and other nucleic acid elements involved in the analysis of genomic DNA. In certain aspects, a nucleic acid comprises a wild-type, a mutant, or a polymorphic nucleic acid.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length. A “gene” refers to coding sequence of a gene product, as well as introns and the promoter of the gene product. In addition to the ABCC2 gene, other regulatory regions such as enhancers for ABCC2 are contemplated as nucleic acids for use with compositions and methods of the claimed invention.

In some embodiments, nucleic acids of the invention comprise or are complementary to all or 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 or more contiguous nucleotides, or any range derivable therein, of SEQ ID NO: 1 (ABCC2 cDNA), SEQ ID NO:3 (ABCC2 exon 28); SEQ ID NO:4 (majority of UGT1A1 gene, including nucleotides 169,831 to 187,313 of the UGT1 gene locus with nucleotide 1645 of SEQ ID NO:4 corresponding to nucleotide −3565 from the transcriptional start of the UGT1A1 gene, thus the transcriptional start is located at nucleotide 5212 of SEQ ID NO:4); SEQ ID NO:5-8 (TA repeats in UGT1A1 promoter); SEQ ID NO:9 (SLC01B1 gene); SEQ ID NO:10 (ABCC2 5′ upstream sequence); and/or SEQ ID NO:11 (portion of genomic ABCC2 gene including intron 27).

Moreover, it is contemplated that nucleic acids of the invention may be or be at least 70, 75, 80, 85, 90, 95, 96, 97, 98, 99, or 100% homologous to all or part (any lengths discussed in previous paragraph) of SEQ ID NO:1, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and/or SEQ ID NO:11. One of skill in the art knows how to design and use primers and probes for hybridization and amplification, including the limits of homology needed to implement primers and probes.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss”, a double stranded nucleic acid by the prefix “ds”, and a triple stranded nucleic acid by the prefix “ts.”

In particular aspects, a nucleic acid encodes a protein, polypeptide, or peptide. In certain embodiments, the present invention concerns novel compositions comprising at least one proteinaceous molecule. As used herein, a “proteinaceous molecule,” “proteinaceous composition,” “proteinaceous compound,” “proteinaceous chain,” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

1. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemical synthesis using phosphotriester, phosphite or phosphoramidite chemistry and solid phase techniques such as described in European Patent 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

2. Purification of Nucleic Acids

A nucleic acid may be purified on polyacrylamide gels, cesium chloride centrifugation gradients, chromatography columns or by any other means known to one of ordinary skill in the art (see for example, Sambrook et al., 2001, incorporated herein by reference). In some aspects, a nucleic acid is a pharmacologically acceptable nucleic acid. Pharmacologically acceptable compositions are known to those of skill in the art, and are described herein.

In certain aspects, the present invention concerns a nucleic acid that is an isolated nucleic acid. As used herein, the term “isolated nucleic acid” refers to a nucleic acid molecule (e.g. an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

3. Nucleic Acid Segments

In certain embodiments, the nucleic acid is a nucleic acid segment. As used herein, the term “nucleic acid segment,” are fragments of a nucleic acid, such as, for a non-limiting example, those that encode only part of a ABCC2 gene locus or a ABCC2 gene sequence. Thus, a “nucleic acid segment” may comprise any part of a gene sequence, including from about 2 nucleotides to the full length gene including promoter regions to the polyadenylation signal and any length that includes all the coding region.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:

n to n+y

where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

4. Nucleic Acid Complements

The present invention also encompasses a nucleic acid that is complementary to a nucleic acid. A nucleic acid is “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule. In preferred embodiments, a complement is a hybridization probe or amplification primer for the detection of a nucleic acid polymorphism.

As used herein, the term “complementary” or “complement” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. However, in some diagnostic or detection embodiments, completely complementary nucleic acids are preferred.

### V. Nucleic Acid Detection

Some embodiments of the invention concern identifying polymorphisms in ABCC2, correlating genotype or haplotype to phenotype, wherein the phenotype is altered ABCC2 activity or expression, and then identifying such polymorphisms in patients who have or will be given irinotecan or other drugs or compounds that are ABCC2 substrates. Other embodiments involve polymorphisms in other genes such as the UGT1A4 promoter or encoding region or the SLCO1B1 coding region. Thus, the present invention involves assays for identifying polymorphisms and other nucleic acid detection methods. Nucleic acids, therefore, have utility as probes or primers for embodiments involving nucleic acid hybridization. They may be used in diagnostic or screening methods of the present invention. Detection of nucleic acids encoding ABCC2, UGT1A1, and/or SLCO1B1, as well as nucleic acids involved in the expression or stability of these polypeptides or transcripts, are encompassed by the invention. General methods of nucleic acid detection methods are provided below, followed by specific examples employed for the identification of polymorphisms, including single nucleotide polymorphisms (SNPs).

A. Hybridization

The use of a probe or primer of between 7, 8, 9, 10, 11, 12, 13, 14, 15, 16, 17, 18, 19, 20, 21, 22, 23, 24, 25, 50, 60, 70, 80, 90, or 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting a specific polymorphism. It is generally appreciated that conditions can be rendered more stringent by the addition of increasing amounts of formamide. For example, under highly stringent conditions, hybridization to filter-bound DNA may be carried out in 0.5 M NaHPO₄, 7% sodium dodecyl sulfate (SDS), 1 mM EDTA at 65° C., and washing in 0.1×SSC/0.1% SDS at 68° C. (Ausubel et al., 1989).

Conditions may be rendered less stringent by increasing salt concentration and/or decreasing temperature. For example, a medium stringency condition could be provided by about 0.1 to 0.25M NaCl at temperatures of about 37° C. to about 55° C., while a low stringency condition could be provided by about 0.15M to about 0.9M salt, at temperatures ranging from about 20° C. to about 55° C. Under low stringent conditions, such as moderately stringent conditions the washing may be carried out for example in 0.2×SSC/0.1% SDS at 42° C. (Ausubel et al. 1989). Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 1 0 nM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, calorimetric indicator substrates are known that can be employed to provide a detection means that is visibly or spectrophotometrically detectable, to identify specific hybridization with complementary nucleic acid containing samples. In other aspects, a particular nuclease cleavage site may be present and detection of a particular nucleotide sequence can be determined by the presence or absence of nucleic acid cleavage.

In general, it is envisioned that the probes or primers described herein will be useful as reagents in solution hybridization, as in PCR, for detection of expression or genotype of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

B. Amplification of Nucleic Acids

Nucleic acids used as a template for amplification may be isolated from cells, tissues or other samples according to standard methodologies (Sambrook et al., 2001). In certain embodiments, analysis is performed on whole cell or tissue homogenates or biological fluid samples with or without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to the ABCC2 gene locus (GenBank accession NT030059, incorporated herein by reference), or variants thereof, and fragments thereof are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids that contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected, analyzed or quantified. In certain applications, the detection may be performed by visual means. In certain applications, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Inis et al., 1988, each of which is incorporated herein by reference in their entirety.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA) (described in further detail below), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, Great Britain Application 2 202 328, and in PCT Application PCT/US89/01025, each of which is incorporated herein by reference in its entirety. Qbeta Replicase, described in PCT Application PCT/US87/00880, may also be used as an amplification method in the present invention.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

C. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by spin columns and/or chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized, with or without separation. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

D. Other Assays

Other methods for genetic screening may be used within the scope of the present invention, for example, to detect mutations in genomic DNA, cDNA and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR™ (see above), single-strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substitution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

E. Specific Examples of SNP Screening Methods

Spontaneous mutations that arise during the course of evolution in the genomes of organisms are often not immediately transmitted throughout all of the members of the species, thereby creating polymorphic alleles that co-exist in the species populations. Often polymorphisms are the cause of genetic diseases. Several classes of polymorphisms have been identified. For example, variable nucleotide type polymorphisms (VNTRs), arise from spontaneous tandem duplications of di- or trinucleotide repeated motifs of nucleotides. If such variations alter the lengths of DNA fragments generated by restriction endonuclease cleavage, the variations are referred to as restriction fragment length polymorphisms (RFLPs). RFLPs are been widely used in human and animal genetic analyses.

Another class of polymorphisms are generated by the replacement of a single nucleotide. Such single nucleotide polymorphisms (SNPs) rarely result in changes in a restriction endonuclease site. Thus, SNPs are rarely detectable restriction fragment length analysis. SNPs are the most common genetic variations and occur once every 100 to 300 bases and several SNP mutations have been found that affect a single nucleotide in a protein-encoding gene in a manner sufficient to actually cause a genetic disease. SNP diseases are exemplified by hemophilia, sickle-cell anemia, hereditary hemochromatosis, late-onset alzheimer disease etc.

In context of the present invention, polymorphic mutations that affect the activity and/or level of the ABCC2 gene product, which is responsible for the transport of numerous compounds across cell membranes, will be determined by a series of screening methods. To do this, a sample (such as blood or other bodily fluid or tissue sample) will be taken from a patient for genotype analysis. The presence or absence of SNPs in ABCC2, UGT1A1 and/or SLCO1B1 will determine the ability of the screened individuals to metabolize irinotecan and other agents that are transported by ABCC2. According to methods provided by the invention, these results will be used to adjust and/or alter the dose of irinotecan or other agent administered to an individual in order to reduce drug side effects.

In one embodiment, the presence of the 3972C>T variant in the ABCC2 gene will be determined. The identification of a T at position 3972 on both alleles would indicate that the patient will be slower to dispose of ABCC2 substrates (e.g., irinotecan) than a patient with a C at position 3972 on one or both alleles. Thus, to minimize drug toxicity, it may be desirable to administer a lower drug dose to the patient having a T at position 3972 on both alleles.

In some embodiments, the methods and compositions of the present invention involve determining the sequence at polymorphic sites in linkage disequilibrium with the sequence at position 3972 of the ABCC2 gene. For example, a common haplotype with the 3972 variant is one that includes two promoter variants (−1549(G>A) and −1019A>G) and a 5′ UTR variant (−24C>T). Another haplotype including the 3972 variant and the −1549 and −1019 promoter variants is also common. Thus, in certain embodiments, the methods and compositions of the present invention comprise detecting one or more of the −1549(G>A), −1019A>G, or −24C>T variants in the ABCC2 gene. Yet another haplotype with the 3972 variant includes the −1549(G>A) promoter variant and an intronic variant in intron 13 (+27C>G). Thus, in certain embodiments, the methods and compositions of the present invention comprise detecting one or both of the −1549(G>A) or +27C>G variants in the ABCC2 gene.

SNPs can be the result of deletions, point mutations and insertions and in general any single base alteration, whatever the cause, can result in a SNP. The greater frequency of SNPs means that they can be more readily identified than the other classes of polymorphisms. The greater uniformity of their distribution permits the identification of SNPs “nearer” to a particular trait of interest. The combined effect of these two attributes makes SNPs extremely valuable. For example, if a particular trait (e.g., inability to efficiently metabolize irinotecan) reflects a mutation at a particular locus, then any polymorphism that is linked to the particular locus can be used to predict the probability that an individual will be exhibit that trait.

Several methods have been developed to screen polymorphisms and some examples are listed below. The reference of Kwok and Chen (2003) and Kwok (2001) provide overviews of some of these methods; both of these references are specifically incorporated by reference.

SNPs relating to ABCC2 can be characterized by the use of any of these methods or suitable modification thereof. Such methods include the direct or indirect sequencing of the site, the use of restriction enzymes where the respective alleles of the site create or destroy a restriction site, the use of allele-specific hybridization probes, the use of antibodies that are specific for the proteins encoded by the different alleles of the polymorphism, or any other biochemical interpretation.

i) DNA Sequencing

The most commonly used method of characterizing a polymorphism is direct DNA sequencing of the genetic locus that flanks and includes the polymorphism. Such analysis can be accomplished using either the “dideoxy-mediated chain termination method,” also known as the “Sanger Method” (Sanger et al., 1975) or the “chemical degradation method,” also known as the “Maxam-Gilbert method” (Maxam et al., 1977). Sequencing in combination with genomic sequence-specific amplification technologies, such as the polymerase chain reaction may be utilized to facilitate the recovery of the desired genes (Mullis et al, 1986; European Patent Application 50,424; European Patent Application. 84,796, European Patent Application 258,017, European Patent Application. 237,362; European Patent Application. 201,184; U.S. Pat. Nos. 4,683,202; 4,582,788; and 4,683,194), all of the above incorporated herein by reference.

ii) Exonuclease Resistance

Other methods that can be employed to determine the identity of a nucleotide present at a polymorphic site utilize a specialized exonuclease-resistant nucleotide derivative (U.S. Pat. 4,656,127). A primer complementary to an allelic sequence immediately 3′-to the polymorphic site is hybridized to the DNA under investigation. If the polymorphic site on the DNA contains a nucleotide that is complementary to the particular exonucleotide-resistant nucleotide derivative present, then that derivative will be incorporated by a polymerase onto the end of the hybridized primer. Such incorporation makes the primer resistant to exonuclease cleavage and thereby permits its detection. As the identity of the exonucleotide-resistant derivative is known one can determine the specific nucleotide present in the polymorphic site of the DNA.

iii) Microsequencing Methods

Several other primer-guided nucleotide incorporation procedures for assaying polymorphic sites in DNA have been described (Komher et al., 1989; Sokolov, 1990; Syvanen 1990; Kuppuswamy et al., 1991; Prezant et al., 1992; Ugozzoll et al., 1992; Nyren et al., 1993). These methods rely on the incorporation of labeled deoxynucleotides to discriminate between bases at a polymorphic site. As the signal is proportional to the number of deoxynucleotides incorporated, polymorphisms that occur in runs of the same nucleotide result in a signal that is proportional to the length of the run (Syvanen et al., 1990).

iv) Extension in Solution

French Patent 2,650,840 and PCT Application WO91/02087 discuss a solution-based method for determining the identity of the nucleotide of a polymorphic site. According to these methods, a primer complementary to allelic sequences immediately 3′-to a polymorphic site is used. The identity of the nucleotide of that site is determined using labeled dideoxynucleotide derivatives which are incorporated at the end of the primer if complementary to the nucleotide of the polymorphic site.

v) Genetic Bit Analysis or Solid-Phase Extension

PCT Application WO92/15712 describes a method that uses mixtures of labeled terminators and a primer that is complementary to the sequence 3′ to a polymorphic site. The labeled terminator that is incorporated is complementary to the nucleotide present in the polymorphic site of the target molecule being evaluated and is thus identified. Here the primer or the target molecule is immobilized to a solid phase.

vi) Oligonucleotide Ligation Assay (OLA)

This is another solid phase method that uses different methodology (Landegren et al., 1988). Two oligonucleotides, capable of hybridizing to abutting sequences of a single strand of a target DNA are used. One of these oligonucleotides is biotinylated while the other is detectably labeled. If the precise complementary sequence is found in a target molecule, the oligonucleotides will hybridize such that their termini abut, and create a ligation substrate. Ligation permits the recovery of the labeled oligonucleotide by using avidin. Other nucleic acid detection assays, based on this method, combined with PCR have also been described (Nickerson et al., 1990). Here PCR is used to achieve the exponential amplification of target DNA, which is then detected using the OLA.

vii) Ligase/Polymerase-Mediated Genetic Bit Analysis

U.S. Pat. No. 5,952,174 describes a method that also involves two primers capable of hybridizing to abutting sequences of a target molecule. The hybridized product is formed on a solid support to which the target is immobilized. Here the hybridization occurs such that the primers are separated from one another by a space of a single nucleotide. Incubating this hybridized product in the presence of a polymerase, a ligase, and a nucleoside triphosphate mixture containing at least one deoxynucleoside triphosphate allows the ligation of any pair of abutting hybridized oligonucleotides. Addition of a ligase results in two events required to generate a signal, extension and ligation. This provides a higher specificity and lower “noise” than methods using either extension or ligation alone and unlike the polymerase-based assays, this method enhances the specificity of the polymerase step by combining it with a second hybridization and a ligation step for a signal to be attached to the solid phase.

viii) Invasive Cleavage Reactions

Invasive cleavage reactions can be used to evaluate cellular DNA for a particular polymorphism. A technology called INVADER® employs such reactions (e.g., de Arruda et al., 2002; Stevens et al, 2003, which are incorporated by reference). Generally, there are three nucleic acid molecules: 1) an oligonucleotide upstream of the target site (“upstream oligo”), 2) a probe oligonucleotide covering the target site (“probe”), and 3) a single-stranded DNA with the target site (“target”). The upstream oligo and probe do not overlap but they contain contiguous sequences. The probe contains a donor fluorophore, such as fluoroscein, and an acceptor dye, such as Dabcyl. The nucleotide at the 3′ terminal end of the upstream oligo overlaps (“invades”) the first base pair of a probe-target duplex. Then the probe is cleaved by a structure-specific 5′ nuclease causing separation of the fluorophore/quencher pair, which increases the amount of fluorescence that can be detected. See Lu et al, 2004.

In some cases, the assay is conducted on a solid-surface or in an array format.

ix) Other Methods To Detect SNPs

Several other specific methods for SNP detection and identification are presented below and may be used as such or with suitable modifications in conjunction with identifying polymorphisms of the ABCC2 gene in the present invention. Several other methods are also described on the SNP web site of the NCBI on the World Wide Web at ncbi.nhn.nih.gov/SNP, incorporated herein by reference.

In a particular embodiment, extended haplotypes may be determined at any given locus in a population, which allows one to identify exactly which SNPs will be redundant and which will be essential in association studies. The latter is referred to as ‘haplotype tag SNPs (htSNPs)’, markers that capture the haplotypes of a gene or a region of linkage disequilibrium. See Johnson et al. (2001) and Ke and Cardon (2003), each of which is incorporated herein by reference, for exemplary methods.

The VDA-assay utilizes PCR amplification of genomic segments by long PCR methods using TaKaRa LA Taq reagents and other standard reaction conditions. The long amplification can amplify DNA sizes of about 2,000-12,000 bp. Hybridization of products to variant detector array (VDA) can be performed by a Affymetrix High Throughput Screening Center and analyzed with computerized software.

A method called Chip Assay uses PCR amplification of genomic segments by standard or long PCR protocols. Hybridization products are analyzed by VDA, Halushka et al. (1999), incorporated herein by reference. SNPs are generally classified as “Certain” or “Likely” based on computer analysis of hybridization patterns. By comparison to alternative detection methods such as nucleotide sequencing, “Certain” SNPs have been confirmed 100% of the time; and “Likely” SNPs have been confirmed 73% of the time by this method.

Other methods simply involve PCR amplification following digestion with the relevant restriction enzyme. Yet others involve sequencing of purified PCR products from known genomic regions.

In yet another method, individual exons or overlapping fragments of large exons are PCR-amplified. Primers are designed from published or database sequences and PCR-amplification of genomic DNA is performed using the following conditions: 200 ng DNA template, 0.5 μM each primer, 80 μM each of dCTP, dATP, dTTP and dGTP, 5% formamide, 1.5 mM MgCl₂, 0.5 U of Taq polymerase and 0.1 volume of the Taq buffer. Thermal cycling is performed and resulting PCR-products are analyzed by PCR-single strand conformation polymorphism (PCR-SSCP) analysis, under a variety of conditions, e.g., 5 or 10% polyacrylamide gel with 15% urea, with or without 5% glycerol. Electrophoresis is performed overnight. PCR-products that show mobility shifts are reamplified and sequenced to identify nucleotide variation.

In a method called CGAP-GAI (DEMIGLACE), sequence and alignment data (from a PHRAP.ace file), quality scores for the sequence base calls (from PHRED quality files), distance information (from PHYLIP dnadist and neighbour programs) and base-calling data (from PHRED ‘−d’ switch) are loaded into memory. Sequences are aligned and examined for each vertical chunk (‘slice’) of the resulting assembly for disagreement. Any such slice is considered a candidate SNP (DEMIGLACE). A number of filters are used by DEMIGLACE to eliminate slices that are not likely to represent true polymorphisms. These include filters that: (i) exclude sequences in any given slice from SNP consideration where neighboring sequence quality scores drop 40% or more; (ii) exclude calls in which peak amplitude is below the fifteenth percentile of all base calls for that nucleotide type; (iii) disqualify regions of a sequence having a high number of disagreements with the consensus from participating in SNP calculations; (iv) removed from consideration any base call with an alternative call in which the peak takes up 25% or more of the area of the called peak; (v) exclude variations that occur in only one read direction. PHRED quality scores were converted into probability-of-error values for each nucleotide in the slice. Standard Baysian methods are used to calculate the posterior probability that there is evidence of nucleotide heterogeneity at a given location.

In a method called CU-RDF (RESEQ), PCR amplification is performed from DNA isolated from blood using specific primers for each SNP, and after typical cleanup protocols to remove unused primers and free nucleotides, direct sequencing using the same or nested primers.

In a method called DEBNICK (METHOD-B), a comparative analysis of clustered EST sequences is performed and confirmed by fluorescent-based DNA sequencing. In a related method, called DEBNICK (METHOD-C), comparative analysis of clustered EST sequences with phred quality >20 at the site of the mismatch, average phred quality >=20 over 5 bases 5′-FLANK and 3′ to the SNP, no mismatches in 5 bases 5′ and 3′ to the SNP, at least two occurrences of each allele is performed and confirmed by examining traces.

In a method identified by ERO (RESEQ), new primers sets are designed for electronically published STSs and used to amplify DNA from 10 different mouse strains. The amplification product from each strain is then gel purified and sequenced using a standard dideoxy, cycle sequencing technique with ³³P-labeled terminators. All the ddATP terminated reactions are then loaded in adjacent lanes of a sequencing gel followed by all of the ddGTP reactions and so on. SNPs are identified by visually scanning the radiographs.

In another method identified as ERO (RESEQ-HT), new primers sets are designed for electronically published murine DNA sequences and used to amplify DNA from 10 different mouse strains. The amplification product from each strain is prepared for sequencing by treating with Exonuclease I and Shrimp Alkaline Phosphatase. Sequencing is performed using ABI Prism Big Dye Terminator Ready Reaction Kit (Perkin-Elmer) and sequence samples are run on the 3700 DNA Analyzer (96 Capillary Sequencer).

FGU-CBT (SCA2-SNP) identifies a method where the region containing the SNP were PCR amplified using the primers SCA2-FP3 and SCA2-RP3. Approximately 100 ng of genomic DNA is amplified in a 50 ml reaction volume containing a final concentration of 5 mM Tris, 25 mM KCl, 0.75 mM MgCl₂, 0.05% gelatin, 20 μmol of each primer and 0.5 U of Taq DNA polymerase. Samples are denatured, annealed and extended and the PCR product is purified from a band cut out of the agarose gel using, for example, the QIAquick gel extraction kit (Qiagen) and is sequenced using dye terminator chemistry on an ABI Prism 377 automated DNA sequencer with the PCR primers.

In a method identified as JBLACK (SEQ/RESTRICT), two independent PCR reactions are performed with genomic DNA. Products from the first reaction are analyzed by sequencing, indicating a unique FspI restriction site. The mutation is confirmed in the product of the second PCR reaction by digesting with Fsp I.

In a method described as KWOK(1), SNPs are identified by comparing high quality genomic sequence data from four randomly chosen individuals by direct DNA sequencing of PCR products with dye-terminator chemistry (see Kwok et al., 1996). In a related method identified as KWOK(2) SNPs are identified by comparing high quality genomic sequence data from overlapping large-insert clones such as bacterial artificial chromosomes (BACs) or P1-based artificial chromosomes (PACs). An STS containing this SNP is then developed and the existence of the SNP in various populations is confirmed by pooled DNA sequencing (see Taillon-Miller et al., 1998). In another similar method called KWOK(3), SNPs are identified by comparing high quality genomic sequence data from overlapping large-insert clones BACs or PACs. The SNPs found by this approach represent DNA sequence variations between the two donor chromosomes but the allele frequencies in the general population have not yet been determined. In method KWOK(5), SNPs are identified by comparing high quality genomic sequence data from a homozygous DNA sample and one or more pooled DNA samples by direct DNA sequencing of PCR products with dye-terminator chemistry. The STSs used are developed from sequence data found in publicly available databases. Specifically, these STSs are amplified by PCR against a complete hydatidiform mole (CHM) that has been shown to be homozygous at all loci and a pool of DNA samples from 80 CEPH parents (see Kwok et al., 1994).

In another such method, KWOK (OverlapSnpDetectionWithPolyBayes), SNPs are discovered by automated computer analysis of overlapping regions of large-insert human genomic clone sequences. For data acquisition, clone sequences are obtained directly from large-scale sequencing centers. This is necessary because base quality sequences are not present/available through GenBak. Raw data processing involves analyzed of clone sequences and accompanying base quality information for consistency. Finished (‘base perfect’, error rate lower than 1 in 10,000 bp) sequences with no associated base quality sequences are assigned a uniform base quality value of 40 (1 in 10,000 bp error rate). Draft sequences without base quality values are rejected. Processed sequences are entered into a local database. A version of each sequence with known human repeats masked is also stored. Repeat masking is performed with the program “MASKERAID.” Overlap detection: Putative overlaps are detected with the program “WUBLAST.” Several filtering steps followed in order to eliminate false overlap detection results, i.e. similarities between a pair of clone sequences that arise due to sequence duplication as opposed to true overlap. Total length of overlap, overall percent similarity, number of sequence differences between nucleotides with high base quality value “high-quality mismatches.” Results are also compared to results of restriction fragment mapping of genomic clones at Washington University Genome Sequencing Center, finisher's reports on overlaps, and results of the sequence contig building effort at the NCBI. SNP detection: Overlapping pairs of clone sequence are analyzed for candidate SNP sites with the ‘POLYBAYES’ SNP detection software. Sequence differences between the pair of sequences are scored for the probability of representing true sequence variation as opposed to sequencing error. This process requires the presence of base quality values for both sequences. High-scoring candidates are extracted. The search is restricted to substitution-type single base pair variations. Confidence score of candidate SNP is computed by the POLYBAYES software.

In method identified by KWOK (TaqMan assay), the TaqMan assay is used to determine genotypes for 90 random individuals. In method identified by KYUGEN(Q1), DNA samples of indicated populations are pooled and analyzed by PLACE-SSCP. Peak heights of each allele in the pooled analysis are corrected by those in a heterozygote, and are subsequently used for calculation of allele frequencies. Allele frequencies higher than 10% are reliably quantified by this method. Allele frequency=0 (zero) means that the allele was found among individuals, but the corresponding peak is not seen in the examination of pool. Allele frequency=0-0.1 indicates that minor alleles are detected in the pool but the peaks are too low to reliably quantify.

In yet another method identified as KYUGEN (Method1), PCR products are post-labeled with fluorescent dyes and analyzed by an automated capillary electrophoresis system under SSCP conditions (PLACE-SSCP). Four or more individual DNAs are analyzed with or without two pooled DNA (Japanese pool and CEPH parents pool) in a series of experiments. Alleles are identified by visual inspection. Individual DNAs with different genotypes are sequenced and SNPs identified. Allele frequencies are estimated from peak heights in the pooled samples after correction of signal bias using peak heights in heterozygotes. For the PCR primers are tagged to have 5′-ATT or 5′-GTT at their ends for post-labeling of both strands. Samples of DNA (10 ng/ul) are amplified in reaction mixtures containing the buffer (10 mM Tris-HCl, pH 8.3 or 9.3, 50 mM KCl, 2.0 mM MgCl₂), 0.25 μM of each primer, 200 μM of each dNTP, and 0.025 units/41 of Taq DNA polymerase premixed with anti-Taq antibody. The two strands of PCR products are differentially labeled with nucleotides modified with R110 and R6G by an exchange reaction of Klenow fragment of DNA polymerase I. The reaction is stopped by adding EDTA, and unincorporated nucleotides are dephosphorylated by adding calf intestinal alkaline phosphatase. For the SSCP: an aliquot of fluorescently labeled PCR products and TAMRA-labeled internal markers are added to deionized formamide, and denatured. Electrophoresis is performed in a capillary using an ABI Prism 310 Genetic Analyzer. Genescan softwares (P-E Biosystems) are used for data collection and data processing. DNA of individuals (two to eleven) including those who showed different genotypes on SSCP are subjected for direct sequencing using big-dye terminator chemistry, on ABI Prism 310 sequencers. Multiple sequence trace files obtained from ABI Prism 310 are processed and aligned by Phred/Phrap and viewed using Consed viewer. SNPs are identified by PolyPhred software and visual inspection.

In yet another method identified as KYUGEN (Method2), individuals with different genotypes are searched by denaturing HPLC (DHPLC) or PLACE-SSCP (Inazuka et al., 1997) and their sequences are determined to identify SNPs. PCR is performed with primers tagged with 5′-ATT or 5′-GTT at their ends for post-labeling of both strands. DHPLC analysis is carried out using the WAVE DNA fragment analysis system (Transgenomic). PCR products are injected into DNASep column, and separated under the conditions determined using WAVEMaker program (Transgenomic). The two strands of PCR products that are differentially labeled with nucleotides modified with R110 and R6G by an exchange reaction of Klenow fragment of DNA polymerase I. The reaction is stopped by adding EDTA, and unincorporated nucleotides are dephosphorylated by adding calf intestinal alkaline phosphatase. SSCP followed by electrophoresis is performed in a capillary using an ABI Prism 310 Genetic Analyzer. Genescan softwares (P-E Biosystems). DNA of individuals including those who showed different genotypes on DHPLC or SSCP are subjected for direct sequencing using big-dye terminator chemistry, on ABI Prism 310 sequencer. Multiple sequence trace files obtained from ABI Prism 310 are processed and aligned by Phred/Phrap and viewed using Consed viewer. SNPs are identified by PolyPhred software and visual inspection. Trace chromatogram data of EST sequences in Unigene are processed with PHRED. To identify likely SNPs, single base mismatches are reported from multiple sequence alignments produced by the programs PHRAP, BRO and POA for each Unigene cluster. BRO corrected possible misreported EST orientations, while POA identified and analyzed non-linear alignment structures indicative of gene mixing/chimeras that might produce spurious SNPs. Bayesian inference is used to weigh evidence for true polymorphism versus sequencing error, misalignment or ambiguity, misclustering or chimeric EST sequences, assessing data such as raw chromatogram height, sharpness, overlap and spacing; sequencing error rates; context-sensitivity; cDNA library origin, etc.

In method identified as MARSHFIELD (Method-B), overlapping human DNA sequences which contained putative insertion/deletion polymorphisms are identified through searches of public databases. PCR primers which flanked each polymorphic site are selected from the consensus sequences. Primers are used to amplify individual or pooled human genomic DNA. Resulting PCR products are resolved on a denaturing polyacrylamide gel and a Phosphorimager is used to estimate allele frequencies from DNA pools.

6. Linkage Disequilibrium

Polymorphisms in linkage disequilibrium with the polymorphism at 3972 of the ABCC2 gene locus may also be used with the methods of the present invention. “Linkage disequilibrium” (“LD” as used herein, though also referred to as “LED” in the art) refers to a situation where a particular combination of alleles (i.e., a variant form of a given gene) or polymorphisms at two loci appears more frequently than would be expected by chance. “Significant” as used in respect to linkage disequilibrium, as determined by one of skill in the art, is contemplated to be a statistical p or a value that may be 0.25 or 0.1 and may be 0.1, 0.05. 0.001, 0.00001 or less. The relationship between ABCC2 haplotypes and the AUC of ABCC2 substrates may be used to correlate the genotype (i.e., the genetic make up of an organism) to a phenotype (i.e., the physical traits displayed by an organism or cell). “Haplotype” is used according to its plain and ordinary meaning to one skilled in the art. It refers to a collective genotype of two or more alleles or polymorphisms along one of the homologous chromosomes.

A common haplotype with the 3972 variant includes two promoter variants (−1549(G>A) and −1019A>G) and a 5′UTR variant (−24C>T). This is found at a frequency of 17.3% in Caucasian, 4.3% in African-Americans, and 10.3% in Asian populations. The 3972 variant is found alone at a frequency of 5.2% in Caucasians and 4.6% in African-Americans. A haplotype including the 3972 variant and the −1549 and −1019 promoter variants has a frequency of 9.2% in Caucasians, and 3.7% in African-Americans. Another haplotype with the 3972 variant includes the −1549(G>A) promoter variant and an intronic variant in intron 13 (+27C>G). This haplotype is found at a frequency of 4.8% in African-Americans.

### VI. Formulations and Dosages

Irinotecan is also known as CPT-11 and it is commercially available as CAMPTOSAR®. CAMPTOSAR® is supplied as a sterile solution in two single-dose sizes: 2-mL vials containing 40 mg irinoteccan hydrochloride and 5-mL vials containing 100 mg irinotecan hydrochloride. Irinotecan hydrochloride is a semisynthetic derivative of camptothecin, which is an alkaloid extract from plants including Camptotheca acuminata.

CAMPTOSAR® Injection can be administered as a monotherapy, but in some instances is indicated as one agent of a first-line therapy to treat colon or rectal cancer. It has been used in combination with 5-fluorouracil (5-FU) and leucovorin. In some cases, this combination treatment is indicated for patient with recurrent or progressed cancer, after they have undergone a fluorouracil-based therapy.

It can be adminstered by intravenous infusion. Dosages of CAMPTOSAR® include 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 300, 305, 310, 315, 320, 325, 330, 335, 340, 345, 350, 355, 360, 365, 370, 375, 380, 385, 390, 400 or more mg/m² on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 26, 37 or on a weekly regimen, such as every 1, 2, 3, 4 weeks or more for 1, 2, 3, 4, 5, 6, 7, 8, 9, 10, 11, 12, 13, 14, 15, 16, 17, 18, 19, 20 or more consecutive or non-consecutive weeks. It is contemplated that dosages can be adjusted to be less than or more than the concentrations discussed above or less frequently or more frequently than the timing discussed above. It is contemplated treatment cycles may be repeated and that there may be a respite between cycles. One of ordinary skill in the art is familiar with dosages regimens. In one example of a typical regimen for single-agent CAMPTOSAR® treatment, a patient is provided 125 mg/m2 IV over 90 minutes on day 1, 8, 15, 22, then a two week rest before the cycle may be resumed. The overall amount of the drug administered to the patient in a single regimen or for the treatment overall may be increased or decreased by about, by at least about, or by at most about 100, 200, 300, 400, 500, 600, 700, 800, 900, 1000, 1100, 1200, 1300, 1400, 1500, 1600, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000 mg/m² or any ranges derivable therein.

The dosages of other ABCC2 drug substrates (drugs are included in Table 1) that are administered to patients is well known to those of skill in the art. These dosages may be reduced or increased relative to a dosage that would have been adminstered in the absence of genotyping. It is specifically contemplated that the dosages of any of those drugs may be similarly altered or modified based on genotypic analysis described herein.

### VII. Kits

Any of the compositions described herein may be comprised in a kit. In a non-limiting example, reagents for determining the genotype of one or both ABCC2 genes are included in a kit. The kit may further include individual nucleic acids that can be used to amplify and/or detect particular nucleic acid sequences of the ABCC2 gene. It may also include one or more buffers, such as a DNA isolation buffers, an amplification buffer or a hybridization buffer. The kit may also contain compounds and reagents to prepare DNA templates and isolate DNA from a sample. The kit may also include various labeling reagents and compounds.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

A kit will also include instructions for employing the kit components as well the use of any other reagent not included in the kit. Instructions may include variations that can be implemented.

It is contemplated that such reagents are embodiments of kits of the invention. Such kits, however, are not limited to the particular items identified above and may include any reagent used directly or indirectly in the detection of polymorphisms in the ABCC2 gene, particularly the 3972C>T polymorphism. Kits include, in some embodiments, nucleic acids capable of amplifying or of probing for a polymorphism in the ABCC2 gene, the UGT1A1 gene, and/or the SLCO1B1 gene. Such kits can include reagents for identifying multiple polymorphisms, and in some embodiments, are directed to identifying one or more haplotypes. The polymorphisms may be in the ABCC2 gene, the UGT1A1 gene, and/or the SLCO1B1 gene.

Kits may include the nucleic acid compositions discussed above with respect to relevant SEQ ID NOs. A person of ordinary skill in the art would be able to discern nucleic acids that could be used in methods of the invention and compositions of kit components based on the description above.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Correlation of the 3972C>T Variant of ABCC2 with Irinotecan Pharmacokinetics

Sixty-four adults (48 Caucasians, 10 African-Americans, 4 Hispanics, and 2 others) with refractory solid tumors took part in the pharmacogenetic study. Genotyping of common variants (q>0.10 in individuals of African and Caucasian origin) was performed for the following genes (number of variants in parenthesis): CES-2 (n=2), ABCC1 (n=7), ABCC2 (n=6), ABCB1 (n=8), CYP3A4*1B (n=1), CYP3A5*3 (n=1), UGT1A9 (n=1), and HNF-1α (n=1) (Table 2).

TABLE 2
Genetic variants typed in this study.

| Gene | Location | Position |
|---|---|---|
| CES-2 | 16q22.1 | −363C > G, 5′UTR |
| CES-2 | 16q22.1 | 1361G > A, intron 1 |
| ABCC1 | 16p13.1 | 1062T > C, synonymous |
| ABCC1 | 16p13.1 | 8A > G, intron 9 |
| ABCC1 | 16p13.1 | −48C>, intron 11 |
| ABCC1 | 16p13.1 | 1684T > C, synonymous |
| ABCC1 | 16p13.1 | −30C > G, intron 18 |
| ABCC1 | 16p13.1 | 4002G > A, synonymous |
| ABCC1 | 16p13.1 | 18A > G, intron 30 |
| ABCC2 | 10q24 | −1549(G > A), promoter |
| ABCC2 | 10q24 | −1019A > G, promoter |
| ABCC2 | 10q24 | −24C > T, 5′UTR |
| ABCC2 | 10q24 | 1249G > A, nonsynonymous, Val417Ile |
| ABCC2 | 10q24 | −34T > C, intron 26 |
| ABCC2 | 10q24 | 3972C > T, synonymous |
| ABCB1 | 7q21.1 | −129T > C, 5′UTR |
| ABCB1 | 7q21.1 | −25G > T, intron 4 |
| ABCB1 | 7q21.1 | −44A > G, intron 9 |
| ABCB1 | 7q21.1 | 1236C > T, synonymous |
| ABCB1 | 7q21.1 | 24C > T, intron 13 |
| ABCB1 | 7q21.1 | +38A > G, intron 14 |
| ABCB1 | 7q21.1 | 2677G > T/A, nonsynonymous, Ala893Ser/Thr |
| ABCB1 | 7q21.1 | 3435C > T, synonymous |
| CYP3A4*1B | 7q21.1 | −392A > G, promoter |
| CYP3A5*3 | 7q21.1 | 22893G > A |
| UGT1A9 | 2q37 | −11810T/9T, exon 1, AF297093 |
| HNF1α | 12q24.2 | 79A > C, nonsynonymous I27L, exon 1, |
| | | NM_000545.3 |

Irinotecan, SN-38, SN-38G, and APC AUCs were measured using noncompartmental analysis (WinNonlin) in the 64 patients in the study after a 350 mg/m² IV dose of irinotecan. AUC ratios of SN-38/irinotecan, APC/irinotecan, and SN-38G/SN-38 were also calculated. After visual inspection of the graphical plots of AUC and ratios stratified by genotype, t test analysis was applied to the data showing the possible presence of an inter-genotype difference in irinotecan pharmacokinetics.

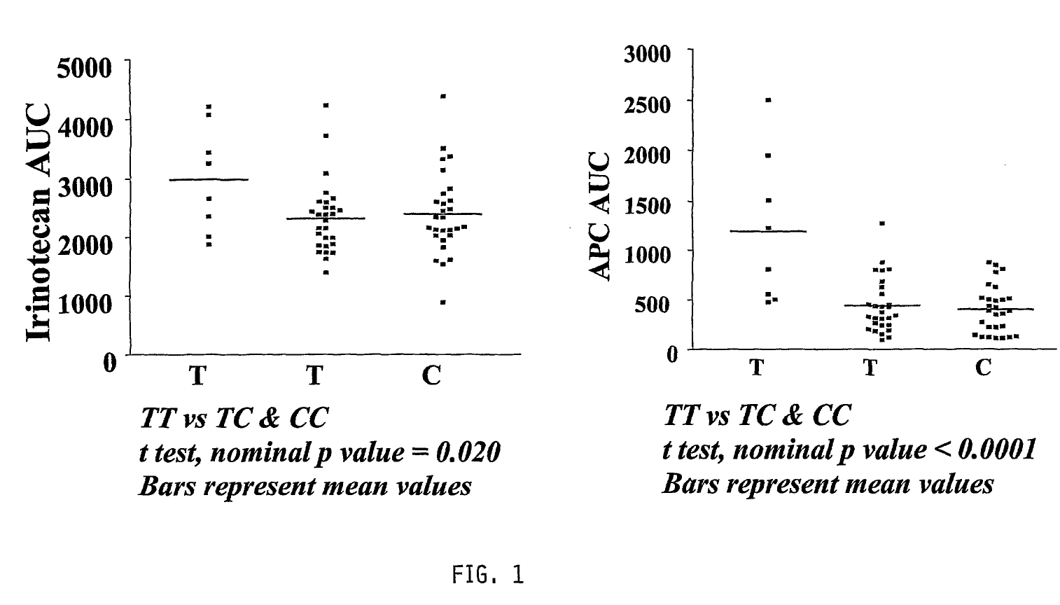

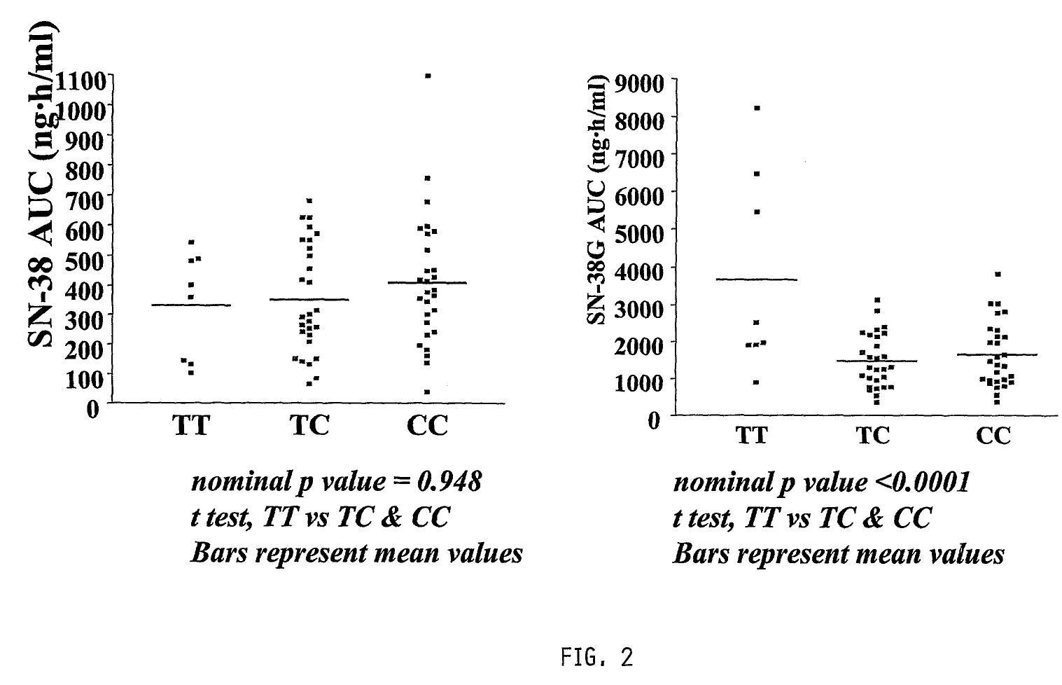

The synonymous 3972C>T (exon 28) in ABCC2 was correlated with irinotecan AUC (p=0.02) (FIG. 1), APC AUC (p=<0.0001) (FIG. 1), and SN-38G AUC (p≦0.001) (FIG. 2), with the TT patients showing higher AUC values compared to CT and CC patients. Higher values of AUC ratios in the TT patients compared to CT and CC patients were also observed in relation to APC/irinotecan (p=<0.0001) and SN-38G/SN-38 (p≦0.001). For SN-38 and SN-38G AUCs, the correlation with 3972C>T was analyzed in patients with 6/6 and 6/7 UGT1A1 genotype (n=54) to avoid confounding effects of 7/7 genotypes. No significant correlation was observed between SN-38 AUC and 3972C>T (p=0.9) (FIG. 2). The frequency of CC, CT, and TT genotypes in the sample population was 0.44, 0.44, and 0.13, respectively. Other gene variants showed either no or borderline statistical significance in the anova test.

### Example 2

### Irinotecan (CPT-11) Pharmacokinetics (PK) and Neutropenia: Interaction Among UGT1A1 and Transporter Genes

In addition to the ABCC2 variants described above, several other ABCC2 variants have been shown to affect ABCC2 expression in vitro. The organic anion transporter polypeptide-1B1 (OATP-1B1, SLCO1B1) is involved in the liver uptake of several compounds. The effects of ABCC2 haplotypes and SLCO1B1 genotypes on CPT-11 PK and neutropenia were evaluated.

Methods: 65 patients previously assessed for pharmacokinetics and toxicity (Innocenti et al., 2004, which is incorporated by reference) were studied. Six SNPs in ABCC2 were genotyped [−1549G>A, −1019A>G, −24C>T, 1249G>A, intron 27 −34C>T, 3972C>T] and haplotypes were estimated. Two SNPs in SLCO1B1 [*1b (388A>G) and *5 (521T>C)] were also genotyped.

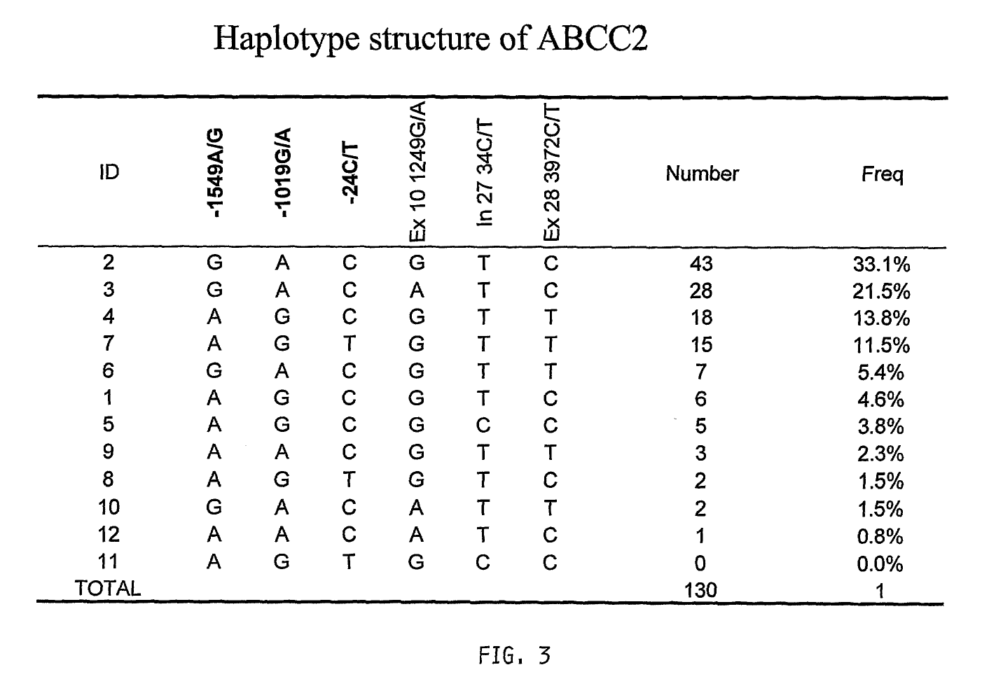

Results: Twelve ABCC2 haplotypes were identified, with haplotypes 2, 3, 4, 7, and 6 having a frequency of 0.33, 0.22, 0.14, 0.12, and 0.05, respectively. See FIG. 3 for haplotypes.

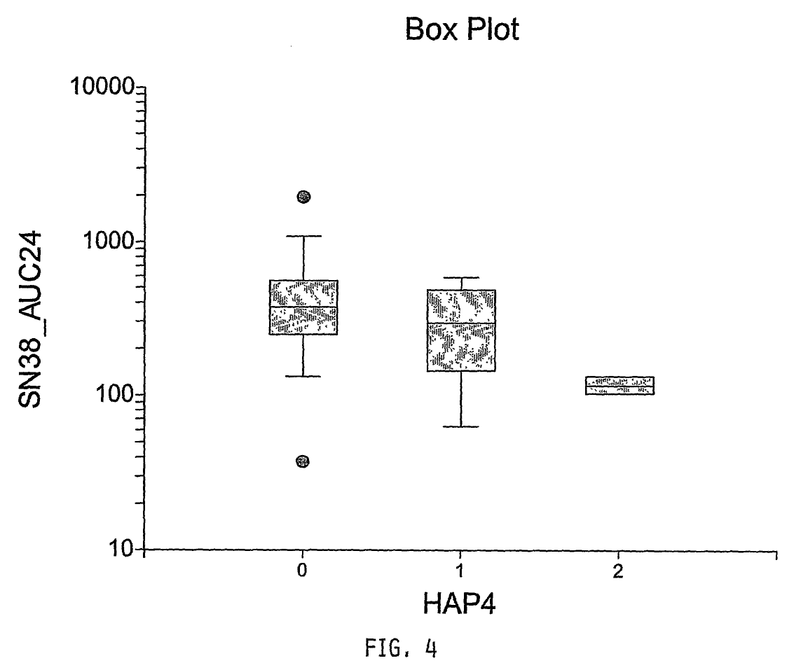

SN-38 AUC v. occurrence of Haplotype 4 was plotted (FIG. 4), indicating the presence of haplotype 4 correlated with toxicity. Moreover, Haplotype 4 was correlated with SN-38G/SN-38 AUC ratios (p<0.0001) in patients. In other words, patients having one haplotype 4 were at higher risk for neutropenia than those not having haploytpe 4, but the risk was lower than those having two of haplotype 4.

SLCO1B1*5 genotype was correlated with SN-38G AUC (p=0.001) and CPT-11 AUC (p<0.0001). Patients with SLCO1B1*5 CT+CC genotype had a higher CPT-11 AUC compared to TT genotype (29.5±8.8 vs. 22.3±5.1 μg*h/ml, p=0.0001). SLCO1B1*1b was associated with an increased ln(ANC nadir), although with borderline significance (p=0.07). The best multivariate model for ln(ANC nadir) included UGT1A1-3156G>A (p=0.03), SLCO1B1*1b (p=0.03), ABCC2 haplotype 4 (p=0.02), total bilirubin (p<0.0001), and gender (p=0.04) (r²=0.49, p<0.0001).

### CONCLUSIONS

SLCO1B1*5 has an effect CPT-11 clearance. SLCO1B1, ABCC2 and UGT1A1 gene variants appear to have additive effects on neutropenia.

### Example 3

### ABCC2 and UGT1A1 Have Additive Effects on Neutropenia and Diarrhea

The indel TA repeats in the UGT1A1 promoter region were combined with ABCC2 haplotype 4 analysis to investigate a correlation with toxicity effects of irinotecan. As shown in FIG. 3, persons with the greatest risk of toxicity had neither a TA repeat of 6 or an ABCC2 haplotype 4. Persons with either an ABCC2 haplotype 4 or six TA repeats in the UGT1A1 gene had the lowest risk for toxicity. Thus, the effects of ABCC2 and UGT1A1 appear additive with respect to diarrhea and neutropenia.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,582,788
U.S. Pat. No. 4,656,127
U.S. Pat. No. 4,659,774
U.S. Pat. No. 4,682,195
U.S. Pat. No. 4,683,194
U.S. Pat. No. 4,683,195
U.S. Pat. No. 4,683,202
U.S. Pat. No. 4,683,202
U.S. Pat. No. 4,683,202
U.S. Pat. No. 4,800,159
U.S. Pat. No. 4,816,571
U.S. Pat. No. 4,883,750
U.S. Pat. No. 4,946,773
U.S. Pat. No. 4,959,463
U.S. Pat. No. 5,141,813
U.S. Pat. No. 5,264,566
U.S. Pat. No. 5,279,721
U.S. Pat. No. 5,428,148
U.S. Pat. No. 5,554,744
U.S. Pat. No. 5,574,146
U.S. Pat. No. 5,602,244
U.S. Pat. No. 5,645,897
U.S. Pat. No. 5,705,629
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,849,481
U.S. Pat. No. 5,849,483
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,770
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,337
U.S. Pat. No. 5,866,366
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,925,525
U.S. Pat. No. 5,928,862
U.S. Pat. No. 5,928,869
U.S. Pat. No. 5,928,870
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 5,952,174
U.S. Pat. No. 6,395,481
U.S. Patent Application No. 20040203034
Ando et al., Cancer Res., 60(24):6921-6926, 2000.
Araki et al., Jpn. J Cancer Res., 84:697-702, 1993.
Ausubel et al., In: Current Protocols in Molecular Biology, Green Pub. Assoc., Inc., and John Wiley & Sons, Inc., NY, (I):2.10.3, 1989.
Beutler et al, Proc. Natl. Acad. Sci. USA, 95(14):8170-8174, 1998.
Borst et al, Biochim. Biophys. Acta, 1461(2):347-357, 1999.
Borst et al. J. Natl. Cancer Inst., 92:1295-1302, 2000.
Bosma et al., N. Eng. J. Med., 333:1171-1175, 1995.
de Ammda et al., Expert. Rev. Mol. Diagn., 2:487-496, 2002.
European Application 329 822
European Application 320 308
European Patent 266,032
European Patent 258,017
European Patent 50,424
European Patent 201,184
European Patent 237,362
European Patent 84,796
French Patent 2,650,840
Froehler et al., Nucleic Acids Res., 14(13):5399-5407, 1986.
Frohman, In: PCR Protocols: A Guide To Methods And Applications, Academic Press, NY, 1990.
Fuchs et al, J. Clin. Oncol., 21(5):807-814, 1993.
Great Britain Patent 2 202 328
Guillamette et al., Cancer Res., 60:950-956, 2000.
Gupta et al., Cancer Res., 54:3723-3725, 1994.
Gupta et al., J. Clin. Oncol., 15:1502-1510, 1997.
Halushka et al., Nat. Genet., 22(3):239-247, 1999.
Inazuka et al., Genome Res, 7(11):1094-1103, 1997.
Innis et al., Proc Natl Acad Sci USA. 85(24):9436-9440, 1988.
Inocenti et al., J. Clin. Oncol., 22(8):1356-59, 2004.
Iyer et al., J. Clin. Invest., 101:847-854, 1998.
Iyer et al., J Phamacogenomics, 2:43-47, 2002.
Johnson et al. Nat. Genet., 29(2):233-237, 2001.
Kaneda et al., Cancer Res., 50:1715-1720, 1990.
Ke and Cardon, Bioinformatics, 19(2):287-288, 2003.
Komher, et al. Nucl. Acids. Res. 17:7779-7784, 1989.
Kuppuswamy, et al. Proc. Natl. Acad. Sci. USA, 88:1143-1147, 1991.
Kwoh et al., Proc. Nat. Acad. Sci. USA, 86:1173, 1989.
Kwok, Annu Rev Genomics Hum Genet., 2:235-58, 2001.
Kwok and Chen, Curr Issues Mol. Biol., April; 5(2):43-60, 2003.
Kwok et al., Genomics, 23(1):138-144, 1994.
Landegren, et al., Science, 241:1077-1080, 1988.
Lu et al., Biopolymers, 73:606-613, 2004.
Maxam, et al., Proc. Natl. Acad. Sci. USA, 74:560, 1977.
Monaghan et al., Lancet, 347:578-581, 1996.
Mullis et al. Cold Spring Harbor Symp. Quant. Biol. 51:263-273, 1986.
Negoro et al., J. Natl. Cancer Inst., 83(16):1164-1168, 1991.
Nickerson et al., Proc. Natl. Acad. Sci. USA, 87:8923-8927, 1990.
Niemi et al., Pharmacogenetics, 7:429-40, 2004.
Nozawa et al., Drug Metab Dispos., 33(3):434-9, 2005.
Nyren et al., Anal. Biochem. 208:171-175, 1993.
Ohara et al., Proc. Natl. Acad. Sci. USA, 86:5673-5677, 1989.
PCT Application PCT/US87/00880
PCT Application PCT/US89/01025
PCT Application WO 88/10315
PCT Application WO 89/06700
PCT Application WO91/02087
PCT Application WO92/15712
Prezant et al., Hum. Mutat. 1:159-164, 1992.
Rothenberg et al., J. Clin. Oncol., 11(11):2194-21204, 1993.
Sambrook et al., In: Molecular Cloning, Cold Spring Harbor Laboratory Press, Cold Spring Harbor, N.Y., 2001.
Sanger, et al., J. Molec. Biol., 94:441, 1975.
Sokolov, Nucl. Acids Res. 18:3671, 1990.
Sparreboom et al., Drug Resist. Update., 3:357-363, 2000.
Stevens et al., Biotechniques, 34:198-203, 2003.
Sugatani et al. Biochem. Biophys. Res. Commun., 292:492-497, 2002.
Sugatani et al., Hepatology, 33:1232-1238, 2001.
Suzuki et al., Semin. Liver. Dis. 18:359-376, 1998.
Suzuki et al., Transporters for bile acids and organic anions, in: G. L. Amidon, W. Sadec (eds.), Membrane Transporters as Drug Targets, Kluwer Academie/Plenum Publishers, New York, 1999.
Syvanen et al., Genomics 8:684-692, 1990.
Taillon-Miller et al., Genoine Res, 8(7):748-754, 1998.
Ugozzoll et al., GATA 9:107-112, 1992.
Vanhoefer et al., J. Clin. Oncol., 19(5):1501-18, 2001.
Walker et al., Proc. Natl. Acad. Sci. USA, 89:392-396, 1992.

1. A method for predicting the level of ABCC2 activity in a patient comprising:
a) determining the sequence at position 3972 in one or both alleles of the ABCC2 gene of the patient, wherein a C at position 3972 on one or both alleles is indicative of a normal level of ABCC2 activity.

2. The method of claim 2, wherein the sequence at position 3972 is determined for both alleles of the ABCC2 gene.

3. The method of claim 2, wherein a T at position 3972 on both alleles of the ABCC2 gene is indicative of a lower than normal level of ABCC2 activity.

4. The method of claim 1, further comprising obtaining a sample from the patient and using the sample to determine the sequence at position 3972.

5. The method of claim 4, wherein determining the sequence at position 3972 is performed by a hybridization assay, an allele specific amplification assay, or a sequencing or microsequencing assay.

6. The method of claim 4, wherein the sample comprises buccal cells, mononuclear cells, or cancer cells.

7. The method of claim 1, wherein the sequence at position 3972 is determined by evaluating the sequence of at least one position in linkage disequilibrium with the sequence at position 3972.

8. The method of claim 7, wherein the position in linkage disequilibrium with the sequence at position 3972 is selected from the group consisting of positions −1549, −1019, −24, and +27.

9. The method of claim 1, further comprising administering an ABCC2 substrate to the patient.

10. The method of claim 1, further comprising analyzing a clearance rate for an ABCC2 substrate.

11. The method of claim 10, wherein the substrate is selected from the group consisting of irinotecan, APC, and SN-38G.

12. The method of claim 1, wherein the patient is a cancer patient and wherein a C at position 3972 on one or both alleles is indicative of a lower probability of an antitumor response to an anticancer agent that is an ABCC2 substrate than the probability if the patient has a T on both alleles at position 3972.

13. The method claim 12, wherein the ABCC2 substrate is irinotecan.

14. The method of claim 12, further comprising administering the anticancer agent to the patient.

15. The method of claim 13, further comprising administering to the patient a second anticancer agent that is not an ABCC2 substrate.

16. The method of claim 12, further comprising prescribing a dosage of the anticancer agent based on determining the sequence at position 3972 in one or both alleles of the ABCC2 gene.

17. A method for determining dosage of an ABCC2 substrate for a patient comprising:
a) determining the sequence at position 3972 in one or both alleles of the ABCC2 gene of the patient, wherein a C at position 3972 on one or both alleles indicates a higher dosage of the substrate than is indicated for a patient with a T at position 3972 in both alleles of the ABCC2 gene.

18. The method of claim 17, further comprising obtaining a sample from the patient and using the sample to determine the sequence at position 3972.

19. The method of claim 18, wherein determining the sequence at position 3972 is performed by a hybridization assay, an allele specific amplification assay, or a sequencing or microsequencing assay.

20. The method of claim 18, wherein the sample comprises buccal cells, mononuclear cells, or cancer cells.

21. The method of claim 17, wherein the sequence at position 3972 is determined by evaluating the sequence of a position in linkage disequilibrium with a sequence at position 3972.

22. The method of claim 17, further comprising prescribing a dosage of the substrate based on determining the sequence at position 3972 in one or both alleles of the ABCC2 gene.

23. A method for predicting risk of irinotecan toxicity in a patient comprising determining:
a) the sequence at position 3972 in one or both alleles of the ABCC2 gene, wherein a C at position 3972 on one or both alleles indicates a lower risk of toxicity than a T at position 3972 in both alleles of the ABCC2 gene;
b) the number, if any, of haplotype 4 in the ABCC2 gene (−1549 A, −1019 G, −24 C, 1249 G, 34 T in intron 27, and 3972 T) of the patient, wherein one allele of haplotype 4 is indicative of a greater risk of toxicity than for a patient having two alleles with haplotype 4 but a lesser risk of toxicity than for a patient having no alleles with haplotype 4; and/or,
c) the sequence in one or both alleles of the SLC01B1 gene at position 388, wherein i) a G in one allele is indicative of a similar or lower risk than an A in one allele, or ii) a G in both alleles is indicative of a lower risk than a G in one allele and an A in the other allele, which is indicative of a lower risk than an A in both alleles.

24. The method of claim 23, wherein the method comprises determining at least two of a), b) or c).

25. The method of claim 23, further comprising determining:
d) the sequence in one or both alleles of the UGT1A1 gene at position −3156, wherein i) a G in one allele is indicative of a similar or lower risk than an A in one allele, or ii) a G in both alleles is indicative of a lower risk than a G in one allele and an A in the other allele, which is indicative of a lower risk than an A in both alleles; and/or,
e) the number of TA repeats in the promoter of the UGT1A1 gene, wherein i) six TA repeats in one allele is indicative of a similar or lower risk than seven TA repeats in one allele, or ii) six TA repeats in both alleles is indicative of a lower risk than six TA repeats in one allele and seven TA repeats in the other allele, which is indicative of a lower risk than seven TA repeats in both alleles.

26. The method of claim 23, further comprising assaying total bilirubin amounts in the patient.

27. The method of claim 23, further comprising obtaining a sample from the patient and using the sample to make any determinations.

28. The method of claim 27, wherein determining any of 1), 2) or 3) is performed by a hybridization assay, an allele specific amplification assay, or a sequencing or microsequencing assay.

29. The method of claim 27, wherein the sample comprises buccal cells, mononuclear cells, or cancer cells.

30. The method of claim 23, wherein a sequence is determined by evaluating the sequence of a position in linkage disequilibrium with it.

31. The method of claim 23, further comprising prescribing a dosage of irinotecan based on determinations of at least 1), 2), and/or 3).

32. A method for predicting a clearance rate for irinotecan in a patient comprising
a) determining the sequence of the patient at either 1) position 3972 in one or both alleles of the ABCC2 gene, wherein a C at position 3972 in one or both alleles is indicative of a normal clearance rate for irinotecan; 2) position 521 in one or both alleles of the SLCO1B1 gene, wherein a C at position 521 in one or both alleles is indicative of a lower clearance rate than a T in both alleles; or 3) both positions 1) and 2).

33. The method of claim 32, wherein the sequence at position 3972 is determined for one or both alleles of the ABCC2 gene.

34. The method of claim 33, wherein a T at position 3972 on both alleles of the ABCC2 gene is indicative of a lower than normal clearance rate for irinotecan.

35. The method of claim 32, further comprising obtaining a sample from the patient and using the sample to determine the sequence at position 3972.

36. The method of claim 35, wherein determining the sequence at position 3972 is performed by a hybridization assay, an allele specific amplification assay, or a sequencing or microsequencing assay.

37. The method of claim 35, wherein the sample comprises buccal cells, mononuclear cells, or cancer cells.

38. The method of claim 32, wherein the sequence at position 3972 is determined by evaluating the sequence of a position in linkage disequilibrium with a sequence at position 3972.

39. The method of claim 32, further comprising administering irinotecan to the patient.

40. The method of claim 38, further comprising prescribing a dosage of irinotecan based on determining the sequence at position 3972 in one or both alleles of the ABCC2 gene.

41. A kit comprising, in suitable container means, at least one nucleic acid for determining the sequence at
a) position 3972, 1549, −1019, −24, 1249, 34 in intron 27, and/or 3972 in an ABCC2 gene; and/or
b) position 388 in a SLC01B1 gene.

42. The kit of claim 41, further comprising at least one nucleic acid for determining:
c) the sequence at position −3156 in a UGT1A1 gene; and/or
d) the number of TA repeats in the UGT1A1 gene promoter.

43. The kit of claim 41, wherein the nucleic acid is a primer for amplifying the sequence(s).

44. The kit of claim 41, wherein the nucleic acid is a specific hybridization probe for detecting the sequence(s).

45. The kit of claim 44, wherein the specific hybridization probe is comprised in an oligonucleotide array or microarray.

46. The kit of claim 41, comprising at least one nucleic acid for determining the sequence of position 3972, 1549, −1019, −24, 1249, 34 in intron 27, and 3972 in an ABCC2 gene (haplotype 4).


SEQUENCE LISTING (11 sequences)

SEQ ID NO: 1 - DNA, 4868 bp, Homo sapiens
  CDS (38)..(4675)
gcggccgcgtctttgttccagacgcagtccaggaatcatgctggagaagttctgcaactctactttttggaattcctcattcctggacagtccggaggcagacctgccactttgttttgagcaaactgttctggtgtggattcccttgggcttcctatggctcctggccccctggcagcttctccacgtgtataaatccaggaccaagagatcctctaccaccaaactctatcttgctaagcaggtattcgttggttttcttcttattctagcagccatagagctggcccttgtactcacagaagactctggacaagccacagtccctgctgttcgatataccaatccaagcctctacctaggcacatggctcctggttttgctgatccaatacagcagacaatggtgtgtacagaaaaactcctggttcctgtccctattctggattctctcgatactctgtggcactttccaatttcagactctgatccggacactcttacagggtgacaattctaatctagcctactcctgcctgttcttcatctcctacggattccagatcctgatcctgatcttttcagcattttcagaaaataatgagtcatcaaataatccatcatccatagcttcattcctgagtagcattacctacagctggtatgacagcatcattctgaaaggctacaagcgtcctctgacactcgaggatgtctgggaagttgatgaagagatgaaaaccaagacattagtgagcaagtttgaaacgcacatgaagagagagctgcagaaagccaggcgggcactccagagacggcaggagaagagctcccagcagaactctggagccaggctgcctggcttgaacaagaatcagagtcaaagccaagatgcccttgtcctggaagatgttgaaaagaaaaaaaagaagtctgggaccaaaaaagatgttccaaaatcctggttgatgaaggctctgttcaaaactttctacatggtgctcctgaaatcattcctactgaagctagtgaatgacatcttcacgtttgtgagtcctcagctgctgaaattgctgatctcctttgcaagtgaccgtgacacatatttgtggattggatatctctgtgcaatcctcttattcactgcggctctcattcagtctttctgccttcagtgttatttccaactgtgcttcaagctgggtgtaaaagtacggacagctatcatggcttctgtatataagaaggcattgaccctatccaacttggccaggaaggagtacaccgttggagaaacagtgaacctgatgtctgtggatgcccagaagctcatggatgtgaccaacttcatgcacatgctgtggtcaagtgttctacagattgtcttatctatcttcttcctatggagagagttgggaccctcagtcttagcaggtgttggggtgatggtgcttgtaatcccaattaatgcgatactgtccaccaagagtaagaccattcaggtcaaaaatatgaagaataaagacaaacgtttaaagatcatgaatgagattcttagtggaatcaagatcctgaaatattttgcctgggaaccttcattcagagaccaagtacaaaacctccggaagaaagagctcaagaacctgctggcctttagtcaactacagtgtgtagtaatattcgtcttccagttaactccagtcctggtatctgtggtcacattttctgtttatgtcctggtggatagcaacaatattttggatgcacaaaaggccttcacctccattaccctcttcaatatcctgcgctttcccctgagcatgcttcccatgatgatctcctccatgctccaggccagtgtttccacagagcggctagagaagtacttgggaggggatgacttggacacatctgccattcgacatgactgcaattttgacaaagccatgcagttttctgaggcctcctttacctgggaacatgattcggaagccacagtccgagatgtgaacctggacattatggcaggccaacttgtggctgtgataggccctgtcggctctgggaaatcctccttgatatcagccatgctgggagaaatggaaaatgtccacgggcacatcaccatcaagggcaccactgcctatgtcccacagcagtcctggattcagaatggcaccataaaggacaacatcctttttggaacagagtttaatgaaaagaggtaccagcaagtactggaggcctgtgctctcctcccagacttggaaatgctgcctggaggagatttggctgagattggagagaagggtataaatcttagtgggggtcagaagcagcggatcagcctggccagagctacctaccaaaatttagacatctatcttctagatgaccccctgtctgcagtggatgctcatgtaggaaaacatatttttaataaggtcttgggccccaatggcctgttgaaaggcaagactcgactcttggttacacatagcatgcactttcttcctcaagtggatgagattgtagttctggggaatggaacaattgtagagaaaggatcctacagtgctctcctggccaaaaaaggagagtttgctaagaatctgaagacatttctaagacatacaggccctgaagaggaagccacagtccatgatggcagtgaagaagaagacgatgactatgggctgatatccagtgtggaagagatccccgaagatgcagcctccataaccatgagaagagagaacagctttcgtcgaacacttagccgcagttctaggtccaatggcaggcatctgaagtccctgagaaactccttgaaaactcggaatgtgaatagcctgaaggaagacgaagaactagtgaaaggacaaaaactaattaagaaggaattcatagaaactggaaaggtgaagttctccatctacctggagtacctacaagcaataggattgttttcgatattcttcatcatccttgcgtttgtgatgaattctgtggcttttattggatccaacctctggctcagtgcttggaccagtgactctaaaatcttcaatagcaccgactatccagcatctcagagggacatgagagttggagtctacggagctctgggattagcccaaggtatatttgtgttcatagcacatttctggagtgcctttggtttcgtccatgcatcaaatatcttgcacaagcaactgctgaacaatatccttcgagcacctatgagattttttgacacaacacccacaggccggattgtgaacaggtttgccggcgatatttccacagtggatgacaccctgcctcagtccttgcgcagctggattacatgcttcctggggataatcagcacccttgtcatgatctgcatggccactcctgtcttcaccatcatcgtcattcctcttggcattatttatgtatctgttcagatgttttatgtgtctacctcccgccagctgaggcgtctggactctgtcaccaggtccccaatctactctcacttcagcgagaccgtatcaggtttgccagttatccgtgcctttgagcaccagcagcgatttctgaaacacaatgaggtgaggattgacaccaaccagaaatgtgtcttttcctggatcacctccaacaggtggcttgcaattcgcctggagctggttgggaacctgactgtcttcttttcagccttgatgatggttatttatagagataccctaagtggggacactgttggctttgttctgtccaatgcactcaatatcacacaaaccctgaactggctggtgaggatgacatcagaaatagagaccaacattgtggctgttgagcgaataactgagtacacaaaagtggaaaatgaggcaccctgggtgactgataagaggcctccgccagattggcccagcaaaggcaagatccagtttaacaactaccaagtgcggtaccgacctgagctggatctggtcctcagagggatcacttgtgacatcggtagcatggagaagattggtgtggtgggcaggacaggagctggaaagtcatccctcacaaactgcctcttcagaatcttagaggctgccggtggtcagattatcattgatggagtagatattgcttccattgggctccacgacctccgagagaagctgaccatcatcccccaggaccccatcctgttctctggaagcctgaggatgaatctcgaccctttcaacaactactcagatgaggagatttggaaggccttggagctggctcacctcaagtcttttgtggccagcctgcaacttgggttatcccacgaagtgacagaggctggtggcaacctgagcataggccagaggcagctgctgtgcctgggcagggctctgcttcggaaatccaagatcctggtcctggatgaggccactgctgcggtggatctagagacagacaacctcattcagacgaccatccaaaacgagttcgcccactgcacagtgatcaccatcgcccacaggctgcacaccatcatggacagtgacaaggtaatggtcctagacaacgggaagattatagagtgcggcagccctgaagaactgctacaaatccctggacccttttactttatggctaaggaagctggcattgagaatgtgaacagcacaaaattctagcagaaggccccatgggttagaaaaggactataagaataatttcttatttaattttattttttataaaatacagaatacatacaaaagtgtgtataaaatgtacgttttaaaaaaggataagtgaacacccatgaacctactacccaggttaagaaaataaatgtcaccaggtacttgagaaacccctcgattg

SEQ ID NO: 2 - protein, 1545 aa, Homo sapiens
MLEKFCNSTFWNSSFLDSPEADLPLCFEQTVLVWIPLGFLWLLAPWQLLHVYKSRTKRSSTTKLYLAKQVFVGFLLILAAIELALVLTEDSGQATVPAVRYTNPSLYLGTWLLVLLIQYSRQWCVQKNSWFLSLFWILSILCGTFQFQTLIRTLLQGDNSNLAYSCLFFISYGFQILILIFSAFSENNESSNNPSSIASFLSSITYSWYDSIILKGYKRPLTLEDVWEVDEEMKTKTLVSKFETHMKRELQKARRALQRRQEKSSQQNSGARLPGLNKNQSQSQDALVLEDVEKKKKKSGTKKDVPKSWLMKALFKTFYMVLLKSFLLKLVNDIFTFVSPQLLKLLISFASDRDTYLWIGYLCAILLFTAALIQSFCLQCYFQLCFKLGVKVRTAIMASVYKKALTLSNLARKEYTVGETVNLMSVDAQKLMDVTNFMHMLWSSVLQIVLSIFFLWRELGPSVLAGVGVMVLVIPINAILSTKSKTIQVKNMKNKDKRLKIMNEILSGIKILKYFAWEPSFRDQVQNLRKKELKNLLAFSQLQCVVIFVFQLTPVLVSVVTFSVYVLVDSNNILDAQKAFTSITLFNILRFPLSMLPMMISSMLQASVSTERLEKYLGGDDLDTSAIRHDCNFDKAMQFSEASFTWEHDSEATVRDVNLDIMAGQLVAVIGPVGSGKSSLISAMLGEMENVHGHITIKGTTAYVPQQSWIQNGTIKDNILFGTEFNEKRYQQVLEACALLPDLEMLPGGDLAEIGEKGINLSGGQKQRISLARATYQNLDIYLLDDPLSAVDAHVGKHIFNKVLGPNGLLKGKTRLLVTHSMHFLPQVDEIVVLGNGTIVEKGSYSALLAKKGEFAKNLKTFLRHTGPEEEATVHDGSEEEDDDYGLISSVEEIPEDAASITMRRENSFRRTLSRSSRSNGRHLKSLRNSLKTRNVNSLKEDEELVKGQKLIKKEFIETGKVKFSIYLEYLQAIGLFSIFFIILAFVMNSVAFIGSNLWLSAWTSDSKIFNSTDYPASQRDMRVGVYGALGLAQGIFVFIAHFWSAFGFVHASNILHKQLLNNILRAPMRFFDTTPTGRIVNRFAGDISTVDDTLPQSLRSWITCFLGIISTLVMICMATPVFTIIVIPLGIIYVSVQMFYVSTSRQLRRLDSVTRSPIYSHFSETVSGLPVIRAFEHQQRFLKHNEVRIDTNQKCVFSWITSNRWLAIRLELVGNLTVFFSALMMVIYRDTLSGDTVGFVLSNALNITQTLNWLVRMTSEIETNIVAVERITEYTKVENEAPWVTDKRPPPDWPSKGKIQFNNYQVRYRPELDLVLRGITCDIGSMEKIGVVGRTGAGKSSLTNCLFRILEAAGGQIIIDGVDIASIGLHDLREKLTIIPQDPILFSGSLRMNLDPFNNYSDEEIWKALELAHLKSFVASLQLGLSHEVTEAGGNLSIGQRQLLCLGRALLRKSKILVLDEATAAVDLETDNLIQTTIQNEFAHCTVITIAHRLHTIMDSDKVMVLDNGKIIECGSPEELLQIPGPFYFMAKEAGIENVNSTKF

SEQ ID NO: 3 - DNA, 184 bp, Homo sapiens
aacttacttctcatcttgtctccttgccaggcaccctgggtgactgataagaggcctccgccagattggcccagcaaaggcaagatccagtttaacaactaccaagtgcggtaccgacctgagctggatctggtcctcagagggatcacttgtgacatcggtagcatggagaaggtaggtggag

SEQ ID NO: 4 - DNA, 261 bp, Human immunodeficiency virus type 1
gcaaatctcacagacaatactaaaaccataatagtacagctgaatcaatctgtagaaattaattgcacaagacccagcaacaatacaaggaaaagtatacatataggaccagggaaagcattttatgcaacaggaagcataataggagatataagacaagcacattgtaaccttagtagaacacaatggaataacactttagaacagatagttaaaaaattaagagaacaatttaaaaataaaacaatagcttttaagcac

SEQ ID NO: 5 - DNA, 488 bp, Homo sapiens
caagtgagcaggcagtaccgggggagctgtggagtgggcactcttacaggtttccatggcgaaagcgggggtacagttgtgttcttttctttctaaaaggctttctaaaaagccttctgtttaatttctggaaaagaagcctaacttgttcactacatagtcgtccttcttcctctctggtaacacttgttggtctgtggaaatactaatttaatggatcctgaggttctggaagtactttgctgtgttcactcaagaatgtgatttgagtatgaaattccagccagttcaactgttgttgcctattaagaaacctaataaagctccaccttctttatctctgaaagtgaactccctgctacctttgtggactgacagctttttacagtcacgtgacacagtcaaacattaacttggtgtatcgattggtttttgccatatatatatataagtaggagagggcgaacctctggcaggagcaaaggcgc

SEQ ID NO: 6 - DNA, 490 bp, Homo sapiens
caagtgagcaggcagtaccgggggagctgtggagtgggcactcttacaggtttccatggcgaaagcgggggtacagttgtgttcttttctttctaaaaggctttctaaaaagccttctgtttaatttctggaaaagaagcctaacttgttcactacatagtcgtccttcttcctctctggtaacacttgttggtctgtggaaatactaatttaatggatcctgaggttctggaagtactttgctgtgttcactcaagaatgtgatttgagtatgaaattccagccagttcaactgttgttgcctattaagaaacctaataaagctccaccttctttatctctgaaagtgaactccctgctacctttgtggactgacagctttttatagtcacgtgacacagtcaaacattaacttggtgtatcgattggtttttgccatatatatatatataagtaggagagggcgaacctctggcaggagcaaaggcgc

SEQ ID NO: 7 - DNA, 492 bp, Homo sapiens
caagtgagcaggcagtaccgggggagctgtggagtgggcactcttacaggtttccatggcgaaagcgggggtacagttgtgttcttttctttctaaaaggctttctaaaaagccttctgtttaatttttggaaaagaagcctaacttgttcactacatagtcgtccttcttcctctctggtaacacttgttggtctgtggaaatactaatttaatggatcctgaggttctggaagtactttgctgtgttcactcaagaatgtgatttgagtatgaaattccagccagttcaactgttgttgcctattaagaaacctaataaagctccaccttctttatctctgaaagtgaactccctgctacctttgtggactgacagctttttatagtcacgtgacacagtcaaacattaacttggtgtatcgattggtttttgccatatatatatatatataagtaggagagggcgaacctctggcaggagcaaaggcgc

SEQ ID NO: 8 - DNA, 494 bp, Homo sapiens
caagtgagcaggcagtaccgggggagctgtggagtgggcactcttacaggtttccatggcgaaagcgggggtacagttgtgttcttttctttctaaaaggctttctaaaaagccttctgtttaatttttggaaaagaagcctaacttgttcactacatagtcgtccttcttcctctctggtaacacttgttggtctgtggaaatactaatttaatggatcctgaggttctggaagtactttgctgtgttcactcaagaatgtgatttgagtatgaaattccagccagttcaactgttgttgcctattaagaaacctaataaagctccaccttctttatctctgaaagtgaactccctgctacctttgtggactgacagctttttatagtcacgtgacacagtcaaacattaacttggtgtatcgattggtttttgccatatatatatatatatataagtaggagagggcgaacctctggcaggagcaaaggcgc

SEQ ID NO: 9 - DNA, 2830 bp, Homo sapiens
  CDS (135)..(2210)
cggacgcgtgggcggacgcgtgggtcgcccacgcgtccgacttgttgcagttgctgtaggattctaaatccaggtgattgtttcaaactgagcatcaacaacaaaaacatttgtatgatatctatatttcaatcatggaccaaaatcaacatttgaataaaacagcagaggcacaaccttcagagaataagaaaacaagatactgcaatggattgaagatgttcttggcagctctgtcactcagctttattgctaagacactaggtgcaattattatgaaaagttccatcattcatatagaacggagatttgagatatcctcttctcttgttggttttattgacggaagctttgaaattggaaatttgcttgtgattgtatttgtgagttactttggatccaaactacatagaccaaagttaattggaatcggttgtttcattatgggaattggaggtgttttgactgctttgccacatttcttcatgggatattacaggtattctaaagaaactaatatcgattcatcagaaaattcaacatcgaccttatccacttgtttaattaatcaaattttatcactcaatagagcatcacctgagatagtgggaaaaggttgtttaaaggaatctgggtcatacatgtggatatatgtgttcatgggtaatatgcttcgtggaataggggagactcccatagtaccattggggctttcttacattgatgatttcgctaaagaaggacattcttctttgtatttaggtatattgaatgcaatagcaatgattggtccaatcattggctttaccctgggatctctgttttctaaaatgtacgtggatattggatatgtagatctaagcactatcaggataactcctactgattctcgatgggttggagcttggtggcttaatttccttgtgtctggactattctccattatttcttccataccattctttttcttgccccaaactccaaataaaccacaaaaagaaagaaaagcttcactgtctttgcatgtgctggaaacaaatgatgaaaaggatcaaacagctaatttgaccaatcaaggaaaaaatattaccaaaaatgtgactggttttttccagtcttttaaaagcatccttactaatcccctgtatgttatgtttgtgcttttgacgttgttacaagtaagcagctatattggtgcttttacttatgtcttcaaatacgtagagcaacagtatggtcagccttcatctaaggctaacatcttattgggagtcataaccatacctatttttgcaagtggaatgtttttaggaggatatatcattaaaaaattcaaactgaacaccgttggaattgccaaattctcatgttttactgctgtgatgtcattgtccttttacctattatattttttcatactctgtgaaaacaaatcagttgccggactaaccatgacctatgatggaaataatccagtgacatctcatagagatgtaccactttcttattgcaactcagactgcaattgtgatgaaagtcaatgggaaccagtctgtggaaacaatggaataacttacatctcaccctgtctagcaggttgcaaatcttcaagtggcaataaaaagcctatagtgttttacaactgcagttgtttggaagtaactggtctccagaacagaaattactcagcccatttgggtgaatgcccaagagatgatgcttgtacaaggaaattttacttttttgttgcaatacaagtcttgaatttatttttctctgcacttggaggcacctcacatgtcatgctgattgttaaaattgttcaacctgaattgaaatcacttgcactgggtttccactcaatggttatacgagcactaggaggaattctagctccaatatattttggggctctgattgatacaacgtgtataaagtggtccaccaacaactgtggcacacgtgggtcatgtaggacatataattccacatcattttcaagggtctacttgggcttgtcttcaatgttaagagtctcatcacttgttttatatattatattaatttatgccatgaagaaaaaatatcaagagaaagatatcaatgcatcagaaaatggaagtgtcatggatgaagcaaacttagaatccttaaataaaaataaacattttgtcccttctgctggggcagatagtgaaacacattgttaaggggagaaaaaaagccacttctgcttctgtgtttccaaacagcattgcattgattcagtaagatgttatttttgaggagttcctggtcctttcactaagaatttccacatcttttatggtggaagtataaataagcctatgaacttataataaaacaaactgtaggtagaaaaaatgagagtactcattgttacattatagctacatatttgtggttaaggttagactatatgatccatacaaattaaagtgagagacatggttactgtgtaataaaagaaaaaatacttgttcaggtaattctaattcttaataaaacaaatgagtatcatacaggtagaggttaaaaaggaggagctagattcatatcctaagtaaagagaaatgcctagtgtctattttattaaacaaacaaacacagagtttgaactataatactaaggcctgaagtctagcttggatatatgctacaataatatctgttactcacataaaattatatatttcacagactttatcaatgtataattaacaattatcttgtttaagtaaatttagaatacatttaagtattgtggaagaaataaagacattccaatatttgcaaaaaaaaaaaaaaa

SEQ ID NO: 10 - DNA, 3000 bp, Homo sapiens
  CDS (2897)..(2929)
actcatgaaagtgtggagatcatcccctccttatggctgagtcactctggaatttttctctctcaaacttatctacactgggcctccggcaattcaattacagttcaggttttcctgccctagccctggttcctgctgagatttcttttcttttttcttttttttttttttgagacagtgtctttgttgcccaggctggagtgcagtggcgtgatctcaagtcactgcaagctccgcttcccgggttcacgccattctcctgcctcagcatctccgagtagctgggactacaggcgcccggcaccacgcccggccaattttttgtatttttagtagagacagggtttcactgtggtctcgatctcctgacctcgtgatccgcccgcctcggcctcccaaagtgctgggattacaagcgtgagccaccgcgcccggcccctgctgagatttctgcgccagtaaattgtgatttttgatattctcttgtttatctcttcagtttgccctgtaaccttatctctctacagatctaagaagagttattgatttttctatttgttcagatttttagttattgttaggaaagattcatgacttcctggctccttacttgcttttctcattcaatattccctatgagtacatattttatactttaagcaacttgttttgatgaacatttagattctccaccctctctttttttgattacttagaacgtttaggccagacacgatggctcatgcctgtaatcccagcagtttgggaggccgaggcgaatggatcgcttgagcccaggagtttgaggctctacaaaaaaattaaaaaaagaaaattaggtgttaatccttgaccttatatttctcaaagttttaccaagagttcccccggactccgagtcctaccaccagtgccaagagaagtatgcagcctaaaggtgcaccctgccccaagtgagatctcttagttccacagctgacctttcctggctcttctggtgagtggtatggaaaatgatggagaaactgccatatgttttcataatggtcagaccaatttacatttccatcaacagtatactagggttcccttttctccacacccttgccagcacttatcttgttgtttttttttttttttgtaatagccatcctaataggtatgaggtggtatctaactgtggttttgatttgtgttttccagatgacttgtgaagttgattcagttgtcatatacctgttggccatttatatgtcttctttggaaaagtgtctgttcaagtcctttgcccactttttaatttgttagtgtatgtttgctattgagttgtatgagttccttatagtatgttgtggatattaactcttcatcagttatatggtctgcaaatattttctcccacactgaatgctgccttttcattttttttatgtgtatacttgacttttaaaatcagtaacaaggtccatacaattcacatttggctgatatgcctcttgattctcttgtaatttgtaagttcctttgactcctttccttcttttaatttatttattaaaaaatggaatcatttgcactatagaatctcccacattctggattttgacaattgcattccaatggagtcctttagcatgttcctgtgtttcttataatccggtagttagatctagaatttgaccagatttaaggccaattttttttagctaggatactgcatgggtggttatgttttagctaggataccgcatgggtggttcatgatatcatgaaaaaagcatgttcttaaagcaatttaagtgacagtacaaaaggttgggtcaggtggggcacggtagctcatgcctgcaatcccagccctttgggaggccaaggcagaaggattgttgaagcctggagtttgagaccagcctgggcaacatagtgagaccccgtccctacagaaaacatttttttaattagctgggcttgttggcatgtgcctgtagtcccagctactcaggaagctgaggcaggaagatcgcttgaacccatgaggtcaaggctgcaatgaatcatgatggcaacactgcactctagctcaggcaacagaccaagaccctgtctcaaaaaacaaaaaacaataacaaaaaaagaaaaggttgggtcgatgacagtttctagcgactgatgccaccactctgtttacagaatcacccctctcctctggccctgatgttttatcatgatatcatatttacaatgcctggcaaaggcttcattcccatttggcatactacctctcaggcaaatagaacttttgaaagcctgtattatgtatataacatataggctcacactggataagctattttataacctgacttcttcaaagaaagtttacatcatgtttaaaccatgttttagattctatatttttaattaaaaatctaaggaagaaggatatttcacatttctataaactctaagatcttgcagcagaagcgaaactgcacatttaggggtgcctgcccctctactgatgctgccctttgtgggtcatatgtccttaggaaaatgaaagactgtgcactcttgatttgttggccagctctgttgacatctttcagtggttccttttatgtatggccactcctacagaggcctcttgtactttgggaactggtgagtctccctgtccctagggctttttagtcacatgtccatccactgtttcaatgtaacatgcatctaggcaaggttaacgattaaatggttgggatgaaaggtcatcctttacggagaacatcagaatggtagataattcctgttccactttctttgatgaaacaagtaaagaagaaacaacacaatcatattaatagaagagtcttcgttccagacgcagtccaggaatcatgctggagaagttctgcaactctactttttgggtgagaaattacatttatcttcatattgactcttctcagactcagaacaagtggtagttagttaacttag

SEQ ID NO: 11 - DNA, 1709 bp, Homo sapiens
caatgtgagtttgaaggttgggagtttggtttcgttagtgtgcttattcttaaattaagcctgatgtatactgtggagtctgtgctcttgattccttcttaaacccagagggactaaagaagactgaggttcaaaccctggctccatcttttacccatggacgtattccttactcttcctaagtagacatgaatgccatctagaaaactggacataaatattgaccttagggcttaagaaggcaatttcatatgtttcaatctaattgttccctgtctgcctctttgcttggttctagtgagagaataaataactttcttctctagaacgtagcagtaatcagaaaggctttttgaacaaataattcctcctgtgtttatttttcttagatcgtagaataattggaggtaattctaataaagaacatagagtctgataaaactatagaatgtgtctcactgcagcatccccagcatgactagaggatacatggcaggcatgattcttttctagcacctaagacagtggctggcactaccgtaattattcaatcaatatttgttataaatgagtcatttgaccatctttttgaactaatttttttagttcaaagagatttgcatagttacatgtgggcattatataatagtgctgtcttcattagaagttaataaggcctgtggagcacagtggctgacacctgtaatcccagcactttgggaggccaaagtgggtggatcacctgaggtcaggagtttgagaccagcctggccaacatggtgaaaccccatctctactaaaaatacaaaaaaaattagccaggcatggtgatgagtgtctgtaatcccagctactggggaggctgaggcaggagaatcacttgaacccaggaggcagaggttgtagtgagccaagatcgtgccattgcactccagcctgggcaacagagcaagattccatctcaaaaaaaaaaaaaaagaagttaataaggcccaatgtgttcctgccagggcacctaaaaggactttgttattcactagagtcacttagttccttgtgtcactgccctttcatacccccatttctaggctttttagcccaggacagacaaggcaaatagtcaggaaactattagattgaaccaaaacaaaatgactcaaacaccggcaattacatatgcttcaatctacacaaagtggatactctgagtccccatgattccgtcctctgctttctgtggctccctttctttaaccaccatttgctcaacaagaactctctgtgcatatatgcaaactcctgagtctttttctgaaagaagtttgcatctgagtaatctacagattccctggcctggatcatgcctttccctgattcacaaagaacactaggcatccaggcaggaggcctgagctctgctcccactccacccattgccagactctcaaattccagctttagtcccaaaccactagacactcacattgcccttcgcgtgagcacctaattagtctgcagccattcaatgcactgctcttaattccttccccaaggaggcaaggattgtctttcttacacttttccttactcccttgtagagtccagcacagtgctgggtacaaagtcggcactggattgtccttgtggtttgagtggttgagttggtttctgtgcctatgatgattttcagtcttctggtttttctgtag